(19) United States
(12) Patent Application Publication
(10) Pub. No.: US 2023/0064864 A1
(43) Pub. Date: Mar. 2, 2023

(54) Neuromodulation Using Electrical Stimulation

(71) Applicant: Carnegie Mellon University, Pittsburgh, PA (US)

(72) Inventors: Aryn Gittis, Pittsburgh, PA (US); Teresa Anne Spix, Pittsburgh, PA (US)

(21) Appl. No.: 17/792,919

(22) PCT Filed: Jan. 15, 2021
(86) PCT No.: PCT/US2021/013635

§ 371 Date: Jul. 14, 2022

Related U.S. Application Data
(60) Provisional application No. 62/961,238, filed on Jan. 15, 2020.

(51) Int. Cl. A61N 1/36; A61N 1/05; A61N 1/375

(52) U.S. Cl. CPC A61N 1/36067; A61N 1/0534; A61N 1/36171; A61N 1/37514

(57) ABSTRACT

Provided herein is method of modulating a plurality of neurons in a patient, by stimulating an area of the patient's central nervous system. The stimulation includes alternating first periods when a plurality of pulses of electrical stimulation are delivered and second periods when no pulses of electrical stimulation are delivered. The first periods have a duration of about 100 to about 400 ms and the second periods have a duration of about 500 ms to about 1900 ms. The pulses have a frequency of about 100 Hz to about 250 Hz.

### CROSS REFERENCE TO RELATED APPLICATIONS

The present application claims priority to U.S. Provisional Patent Application No. 62/961,238, filed Jan. 15, 2020, the contents of which are incorporated herein by reference in their entirety.

### STATEMENT REGARDING FEDERALLY SPONSORED RESEARCH

This invention was made with U.S. Government support under R01NS101016 and R01NS104835 awarded by the National Institute of Health (NIH). The U.S. Government has certain rights in the invention.

### BACKGROUND OF THE INVENTION

### Field of the Invention

Disclosed herein are systems and methods for providing neuromodulation using electrical stimulation.

### Description of Related Art

Parkinson's disease is the second most common neurological disorder and affects one million people in the U.S. Current therapies for Parkinson's disease are not ideal for long-term care because such therapies lose efficacy and/or cause side effects over time.

New strategies must be developed to repair, not simply mask, brain dysfunction in Parkinson's disease.

Deep brain stimulation (DBS) is a highly effective treatment for ameliorating Parkinsonian motor symptoms, but its therapeutic effects, based on tonic, high frequency stimulation of the subthalamic nucleus (STN), decay rapidly. Other known DBS methods, such as coordinated reset DBS (CR-DBS), which have attempted to address the problem of short therapeutic effect duration, require multiple electrode leads to be implanted in the STN. Accordingly, new interventions are required.

### SUMMARY OF THE INVENTION

Provided herein is a method of modulating a plurality of neurons in a patient, including stimulating an area of the patient's central nervous system, the stimulation including alternating first periods when a plurality of pulses of electrical stimulation are delivered and second periods when no pulses of electrical stimulation are delivered, wherein the first periods have a duration of about 100 to about 400 ms and the second periods have a duration of about 500 ms to about 1900 ms, and wherein the pulses have a frequency of about 100 Hz to about 250 Hz.

Also provided herein is a method for treating a movement disorder in a patient, including implanting a stimulating electrode lead in the patient's basal ganglia; and stimulating one or more neurons in the basal ganglia with a plurality of pulses of electrical stimulation, the electrical stimulation including alternating first periods when a plurality of electrical pulses are delivered and second periods when no electrical pulses are delivered, wherein the first periods have a duration of about 100 to about 400 ms and the second periods have a duration of about 500 ms to about 1900 ms, and wherein the pulses have a frequency of about 100 Hz to about 250 Hz, thereby improving one or more motor symptoms of the movement disorder.

Also provided herein is a system for modulating a plurality of neurons in a patient, including an electrode lead; a pulse generator in electrical communication with the electrode lead, the pulse generator having at least one processor programmed or configured to cause the pulse generator to deliver stimulation through the electrode lead, wherein the stimulation includes alternating first periods when a plurality of pulses of electrical stimulation are delivered and second periods when no pulses of electrical stimulation are delivered, wherein the first periods have a duration of about 100 to about 400 ms and the second periods have a duration of about 500 ms to about 1900 ms, and wherein the pulses have a frequency of about 100 Hz to about 250 Hz.

Further non-limiting embodiments or aspects are set forth in the following numbered clauses:

Clause 1. A method of modulating a plurality of neurons in a patient, comprising: stimulating an area of the patient's central nervous system, the stimulation comprising alternating first periods when a plurality of pulses of electrical stimulation are delivered and second periods when no pulses of electrical stimulation are delivered, wherein the first periods have a duration of about 100 to about 400 ms and the second periods have a duration of about 500 ms to about 1900 ms, and wherein the pulses have a frequency of about 100 Hz to about 250 Hz.

Clause 2. The method according to clause 1, wherein the area of the patient's central nervous system is the basal ganglia.

Clause 3. The method according to clause 1 or clause 2, wherein the area of the patient's central nervous system is the globus pallidus.

Clause 4. The method according to any of clauses 1-3, wherein the area of the patient's central nervous system is the internal globus pallidus.

Clause 5. The method according to any of clauses 1-4, wherein the area of the patient's central nervous system is the external globus pallidus.

Clause 6. The method according to any of clauses 1-5, wherein the pulses are configured to excite parvalbumin (PV)-expressing neurons and inhibit LIM homeobox 6 (Lhx6)-expressing neurons in the external globus pallidus.

Clause 7. The method according to any of clauses 1-6, wherein the first periods have a duration of about 150 ms to about 250 ms, optionally about 175 ms to about 250 ms.

Clause 8. The method according to any of clauses 1-7, wherein the pulses have a frequency of about 150 Hz to about 175 Hz, optionally about 175 Hz.

Clause 9. The method according to any of clauses 1-8, wherein the first periods have a duration of about 250 ms and the pulses have a frequency of about 175 Hz.

Clause 10. The method according to any of clauses 1-9, wherein the pulses have an amplitude of about 0.1 to about 4 mA.

Clause 11. The method according to any of clauses 1-10, wherein the pulses are biphasic pulses.

Clause 12. The method according to any of clauses 1-11, wherein the patient has a movement disorder selected from Parkinson's Disease, essential tremor, dystonia, and Meige Syndrome.

Clause 13. The method according to any of clauses 1-12, wherein the patient has Parkinson's Disease.

Clause 14. The method according to any of clauses 1-13, wherein the globus pallidus of each hemisphere is stimulated.

Clause 15. A method for treating a movement disorder in a patient, comprising: implanting a stimulating electrode lead in the patient's basal ganglia; and stimulating one or more neurons in the basal ganglia with a plurality of pulses of electrical stimulation, the electrical stimulation comprising alternating first periods when a plurality of electrical pulses are delivered and second periods when no electrical pulses are delivered, wherein the first periods have a duration of about 100 to about 400 ms and the second periods have a duration of about 500 ms to about 1900 ms, and wherein the pulses have a frequency of about 100 Hz to about 250 Hz, thereby improving one or more motor symptoms of the movement disorder.

Clause 16. The method according to clause 15, wherein the stimulating electrode lead is implanted in the patient's globus pallidus.

Clause 17. The method according to clause 15 or clause 16 wherein only a single stimulating electrode lead is used, and the stimulating electrode lead is implanted in the patient's external globus pallidus.

Clause 18. The method according to any of clauses 15-17, wherein only a single stimulating electrode lead is used, and the stimulating electrode lead is implanted in the patient's internal globus pallidus.

Clause 19. The method according to any of clauses 15-18, wherein the pulses are configured to excite parvalbumin (PV)-expressing neurons and inhibit LIM homeobox 6 (Lhx6)-expressing neurons in the patient's external globus pallidus.

Clause 20. The method according to any of clauses 15-19, wherein the first periods have a duration of about 150 ms to about 250 ms, optionally about 175 ms to about 250 ms.

Clause 21. The method according to any of clauses 15-20, wherein the pulses have a frequency of about 150 Hz to about 175 Hz, optionally about 175 Hz.

Clause 22. The method according to any of clauses 15-21, wherein the first periods have a duration of about 250 ms and the pulses have a frequency of about 175 Hz.

Clause 23. The method according to any of clauses 15-22, wherein the pulses have an amplitude of about 0.1 to about 4 mA.

Clause 24. The method according to any of clauses 15-23, wherein the pulses are biphasic pulses.

Clause 25. The method according to any of clauses 15-24, wherein the movement disorder is selected from Parkinson's Disease, essential tremor, dystonia, and Meige Syndrome.

Clause 26. The method according to any of clauses 15-25, wherein the patient has Parkinson's Disease.

Clause 27. The method according to any of clauses 15-26, wherein the stimulation improves one or more of tremor, dyskinesia, and rigidity in the patient.

Clause 28. The method according to any of clauses 15-27, wherein two stimulating electrode leads are implanted, and wherein one electrode lead is implanted in the globus pallidus of each hemisphere.

Clause 29. A system for modulating a plurality of neurons in a patient, comprising: an electrode lead; a pulse generator in electrical communication with the electrode lead, the pulse generator comprising at least one processor programmed or configured to cause the pulse generator to deliver stimulation through the electrode lead, wherein the stimulation comprises alternating first periods when a plurality of pulses of electrical stimulation are delivered and second periods when no pulses of electrical stimulation are delivered, wherein the first periods have a duration of about 100 to about 400 ms and the second periods have a duration of about 500 ms to about 1900 ms, and wherein the pulses have a frequency of about 100 Hz to about 250 Hz.

Clause 30. The system according to clause 29, wherein the pulse generator further includes memory and a communication interface.

Clause 31. The system according to clause 29 or clause 30, wherein the pulse generator further includes a battery.

Clause 32. The system according to any of clauses 29-31, wherein the battery is a rechargeable battery.

Clause 33. The system according to any of clauses 29-32, wherein the pulse generator is configured to be implanted in a patient.

Clause 34. The system according to any of clauses 29-33, wherein the first periods have a duration of about 150 ms to about 250 ms, optionally about 175 ms to about 250 ms.

Clause 35. The system according to any of clauses 29-34, wherein the pulses have a frequency of about 150 Hz to about 175 Hz, optionally about 175 Hz.

Clause 36. The system according to any of clauses 29-35, wherein the first periods have a duration of about 250 ms and the pulses have a frequency of about 175 Hz.

Clause 37. The system according to any of clauses 29-36, wherein the pulses have an amplitude of/intensity of about 0.1 to about 4 mA.

Clause 38. The system according to any of clauses 29-37, wherein the pulses are biphasic pulses.

Clause 39. The system according to any of clauses 29-38, wherein the system includes only one electrode lead or only two electrode leads.

Clause 40. The system according to any of clauses 29-39, wherein the one electrode lead is a segmented electrode lead, or wherein both electrode leads are segmented electrode leads.

### BRIEF DESCRIPTION OF THE DRAWINGS

FIG. 1 shows (panel A) schematic (left top) and brain slice (right top) images depicting placement of the stimulating electrode in the internal capsule (ic) and the recording electrode tip in the external globus pallidus (GPe); Parvalbumin-expressing (PV)-GPe and lim-homeobox 6-expressing (Lhx6)-GPe neurons identified by fluorescence (bottom row); Panel B shows extracellular firing rates recorded from 3 GPe neurons responding to continuous 100 Hz electrical stimulation; Panel C is an example of an extracellular recording before (top) and after (bottom) subtracting stimulus artifacts; Panel D shows firing rate modulation graph depicting each neuron's response (‘Modulation Factor’, x-axis) to 30 s of continuous 100 Hz stimulation, plotted vs. each neuron's baseline firing rate (y-axis); The Modulation factor (MF)=[(FRₛₜᵢₘ−FR_{baseline})/(FRₛₜᵢₘ+FR_{baseline})];

FIG. 2 shows synaptic inputs recruited during electrical stimulation onto PV- and Lhx6-GPe neurons. Panel A shows EPSCs evoked by stimulation at different stimulus intensities in PV-GPe (triangle) and Lhx6-GPe (circle) neurons (n=14 pairs of neurons; N=7 mice; KW, χ² (7)=44.4, p<0.00001, pairwise, *p<0.05, **p<0.001; Mann Whitney U), throughout the figure, values are medians±SEM. Insets show typical EPSCs measured across different stimulus intensities in each cell type; Panel B shows IPSCs evoked by stimulation at different stimulus intensities in PV-GPe (triangle) and Lhx6-GPe (circle) neurons (n=14 pairs of neurons; N=7 mice; KW, χ² (7)=8.45, p=0.004, pairwise, *p<0.05; Mann Whitney U). Insets show typical IPSCs measured across different stimulus intensities in each cell type; Panel C shows Inhibition-excitation ratio (peak amplitudes, I-E Ratio=[IPSC]/[IPSC+EPSC]) at each stimulus intensity (14 pairs of neurons; N=7 animals; KW, χ² (7)=57.4, p<0.00001, pairwise, *p<0.05, **p<0.001; Mann Whitney U); Panel D shows representative synaptic responses to 3 s of 100 Hz stimulation (after stimulation artifact removal) in an Lhx6-GPe (top trace) and PV-GPe neuron (bottom trace). Panel E shows population average IPSCs and EPSCs recorded in each cell type (n=10 pairs of neurons; N=3 mice). (F) Inhibitory (top) and excitatory (bottom) synaptic charge calculated for indicated time bins (Mann Whitney U, *p=0.023);

FIG. 3 shows short bursts of stimulation inhibit Lhx6-GPe neurons and excite PV-GPe neurons. Panel A shows three-part recordings from representative Lhx6-GPe (top, middle) and PV-GPe (bottom) neurons. The bottom shows cell-attached recording of the neuron's firing rate (FR) response to electrical stimulation (100 Hz, 3 s). The middle and top show whole-cell recordings of EPSCs (‘Exc’, middle, Vhold=−70 mV) and IPSCs (‘Inh’, top, Vhold=0 mV) evoked by stimulation. Panel B shows modulation factors (averaged across 5 trials) for each neuron during the indicated time bin (n=14 pairs of neurons; N=3 mice). The lightest shaded symbols are neurons in which firing rate was not significantly modified from baseline (p>0.05, paired t-test FR_{baseline} vs. FRₛₜᵢₘ, Trials 1-5). Horizontal markers show average MFLhx6 and MFPV, error bars: SEM. Panel C shows (top) Average modulation factor for the population of Lhx6-GPe (MFLhx6) and PV-GPe neurons (MFPV) calculated in 1 s time bins across stimulation and (bottom) population Separation Index (PSI)=MFPV−MFLhx6 calculated for each 1 s time bin (KW, χ² (2)=12.0, p=0.003, pairwise, *p<0.01), mean±SEM; Panel D shows rasters of extracellularly recorded firing responses to 1 s, 100 Hz electrical stimulation (5 trials), from representative Lhx6-GPe and PV-GPe neurons. Panel E shows modulation factors (averaged across 5 trials) for each neuron during 1 s, 100 Hz stimulation (n=14 pairs of neurons; N=3 mice), the lightest shaded symbols are neurons in which firing rate was not significantly modified from baseline (p>0.05, paired t-test FR_{baseline} vs. FRₛₜᵢₘ, Trials 1-5). Horizontal markers show average MFLhx6 and MFPV, error bars: SEM;

FIG. 4 shows optogenetic stimulation of STN excites both PV-GPe and Lhx6-GPe neurons. Panel A shows an experimental schematic showing global Chronos-ChR2 expression in the STN. STN fibers in the GPe were optically stimulated through the recording objective during recordings from identified PV-GPe and Lhx6-GPe neurons. Panel B shows rasters of extracellularly recorded firing responses during 1 s, 100 Hz optical stimulation (5 trials), from representative Lhx6-GPe and PV-GPe neurons. Panel C shows modulation factors for each neuron (averaged across 5 trials), plotted against their baseline firing rates (n=17 pairs of neurons; N=3 mice), the lightest shaded symbols are neurons in which firing rate was not significantly modified from baseline (p>0.05, paired t-test FR_{baseline} vs. FRₛₜᵢₘ, Trials 1-5). Horizontal markers show average MFLhx6 and MFPV, error bars: SEM. Panel D shows an experimental schematic; identical to Panel A, but with the addition of a stimulating electrode used to compare a neuron's response to both optical and electrical stimulation. Panel E shows rasters of extracellularly recorded firing responses from a representative Lhx6-GPe neuron responding to optical (top) or electrical (bottom) stimulation (1 s, 100 Hz, 1 mA). Panel F shows modulation factors for each Lhx6-GPe neuron (n=5 neurons; N=2 mice), averaged across 3 trials of optical stimulation (top) or 3 trials of electrical stimulation (bottom). Horizontal markers show population mean MFLhx6. Error bars: SEM. Panel G shows rasters of extracellularly recorded firing responses during 1 s, 100 Hz, 1 mA electrical stimulation (5 trials), before (left) and after (right) application of 10 μM NBQX/50 μM APV to block glutamate receptors. Responses are from a representative Lhx6-GPe neurons (top and a representative PV-GPe (bottom neuron. Panel H shows modulation factors calculated for each neuron in response to 100 Hz, 1 s, 1 mA electrical stimulation, before (top) and after (bottom) applying glutamate blockers (n=5 Lhx6-GPe neurons, n=4 PV-GPe neurons; N=2 mice), lightest shaded symbols are neurons in which firing rate was not significantly modified from baseline (p>0.05, paired t-test FR_{baseline} vs. FRₛₜᵢₘ, Trials 1-5). Horizontal markers show average MF_{Lhx6} and MF_{PV}, error bars: SEM;

FIG. 5 shows cell-type specific inhibition of Lhx6-GPe neurons arises from collaterals of striatonigral neurons. Panel A shows an experimental schematic showing Chronos-ChR2 expression in the striatum. Striatal fibers in the GPe were optically stimulated through the recording objective during recordings from identified PV-GPe and Lhx6-GPe neurons. Panel B shows rasters of extracellularly recorded firing responses during 1 s, 100 Hz optical stimulation (5 trials), from representative Lhx6-GPe and PV-GPe neurons. Panel C shows modulation factors for each neuron (averaged across 5 trials), plotted against their baseline firing rates (n=16 pairs of neurons; N=3 mice), lightest shaded symbols are neurons in which firing rate was not significantly modified from baseline (p>0.05, paired t-test FR_{baseline} vs. FRₛₜᵢₘ, Trials 1-5). Horizontal markers show average MFLhx6 and MFPV, error bars: SEM. Panel D shows (top) fluorescent image of striatopallidal pathway, labeled by viral expression of DIO-GFP in an A2A-Cre mouse and (bottom) fluorescent image of striatonigral pathway, labeled by viral expression of DIO-GFP in a D1-Cre mouse. The approximate placement of the stimulating electrode for electrical stimulation experiments is shown for reference. Panel E shows rasters of extracellularly recorded firing responses during 1 s, 100 Hz optical stimulation of the striatonigral pathway (5 trials), from representative Lhx6-GPe and PV-GPe neurons. Panel F shows modulation factors for each neuron (averaged across 5 trials), plotted against their baseline firing rates (n=27 pairs of neurons; N=4 mice), lightest shaded symbols are neurons in which firing rate was not significantly modified from baseline (p>0.05, paired t-test FR_{baseline} vs. FRₛₜᵢₘ, Trials 1-5). Horizontal markers show average MFLhx6 and MFPV, error bars: SEM;

FIG. 6 shows a gaussian regression modeling process to predict parameter combinations that maximize cell-type population separation induced with electrical stimulation. Panel A shows raw data showing extracellular firing responses from paired PV-GPe and Lhx6-GPe neurons responding to different burst designs. Top, left to right: 20 Hz, 500 ms, 2.5×; 50 Hz, 1 s, 1.0×; 150 Hz, 100 ms, 2.0×. Bottom, left to right: 100 Hz, 250 ms, 1.5×; 50 Hz, 10 s, 1.25×. Panel B shows model-predicted changes in firing rates for PV-GPe neurons (MFPV, left) and Lhx6-GPe neurons (MFLhx6, middle) at a stimulus intensity of 2.0× threshold, generated using the first iteration of data collection. For PV, firing rates increased and for Lhx6, firing rates decreased, with magnitude of change shown by darkness of shading. Duration (x-axis) represented on log 2 scale. Right: Degree to which burst combinations are predicted to segregate the responses of PV-GPe and Lhx6-GPe populations. Population Separation Index (PSI)=MFPV−MFLhx6 for all possible burst combinations at a stimulus intensity of 2.0× threshold. Duration (x-axis) represented on log 2 scale.

FIG. 7 shows model-predicted changes in firing rates for PV-GPe neurons (MFPV, left) and Lhx6-GPe neurons (MFLhx6, middle) at indicated stimulus intensities (0.5×-2.5×), generated using the first+second iteration of data collection. For PV, firing rates increased and for Lhx6, firing rates decreased, with magnitude of change shown by darkness of shading. Right: Degree to which burst combinations are predicted to segregate the responses of PV-GPe and Lhx6-GPe populations. Population Separation Index (PSI)=MFPV−MFLhx6 for all possible burst combinations across stimulus intensities. In all columns, duration (x-axis) represented on log 2 scale.

FIGS. 8A-8C show model-driven optimization of burst parameters for cell-type specific modulation. FIG. 8A, panel A shows selected surface plots (from FIG. 7) showing predicted PSIs from this model for burst combinations delivered at stimulus intensities of 1× and 2.5× threshold. Points indicate burst combinations selected for experimental validation (C-H). FIG. 8A, panel B shows experimentally-measured modulation factors for Lhx6-GPe neurons (MFLhx6) and PV-GPe neurons (MFPV) in response to each burst design, parameters indicated on left. Bars, average MF; error bars: SEM. PSI=MF_{PV}−MF_{Lhx6}. FIGS. 8B and 8C show (left) rasters of extracellularly recorded firing responses from representative PVGPe and Lhx6-GPe neurons responding to each burst design (specified for each row). Shaded boxes denote time bin used to calculate modulation factors, (middle) modulation factors for each neuron (averaged across 5 trials), plotted against their baseline firing rates (n=14 pairs of neurons; N=4 mice), unshaded symbols are neurons in which firing rate was not significantly modified from baseline (p>0.05, paired t-test FR_{baseline} vs. FRₛₜᵢₘ, Trials 1-5). Horizontal markers show average MF_{Lhx6} and MF_{PV}, error bars: SEM, and (right) bar graphs indicating the percentage of PV GPe and Lhx6-GPe neurons that responded with an increase in firing rate (dark bars), decrease in firing rate (light bars), or no significant change in firing rate (open bars) during stimulation;

FIG. 9 shows a schematic representation of a system as described herein according to a non-limiting embodiment or aspect; and

FIG. 10 shows a schematic representation of electrode placement (top right) and results of pilot in vivo experiments in mice comparing electrical stimulation with parameters as described herein against conventional, continuous (tonic) DBS (bottom panel).

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

As used herein, “a” or “an” refers to one or more.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

As used herein the term “about” means the listed value±10%.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible. It will be appreciated that numerous other arrangements are possible. Any known electronic communication protocols and/or algorithms can be used such as, for example, TCP/IP (including HTTP and other protocols), WLAN (including 802.11a/b/g/n and other radio frequency-based protocols and methods), analog transmissions, Global System for Mobile Communications (GSM), 3G/4G/LTE, BLUETOOTH, ZigBee, EnOcean, TransferJet, Wireless USB, and the like known to those of skill in the art.

As used herein, the term “computing device” may refer to one or more electronic devices configured to process data. A computing device can include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, “electrical communication,” for example in the context of transmitting electrical pulses from a pulse generator to an electrode, refers to sending an electrical pulse produced by a pulse generator to a skin surface electrode, an electrode lead, a magnetic coil, or like devices capable of generating electrical current to stimulate a nerve or neuron as described herein, typically through an electrically-conductive lead, such as a wire.

The “intensity” of an electrical pulse is proportional to, and refers to either the voltage or current (e.g., milliAmperes or mA) applied to the nerve or neuron, with an increased intensity being proportional to an increased voltage or an increased current applied to the nerve or neuron. Intensity may be measured as, or proportional to, electrical power, e.g., Watts.

As used herein, the term “patient” can refer to any mammal, including humans, and a “human patient” is any human.

Provided herein are methods for stimulating neurons and systems for performing such stimulations. These methods improve upon known DBS methods in that they provide long-lasting relief from symptoms of movement disorders and do not require multiple electrode placements in, or tonic stimulation of, the central nervous system in order to achieve the long-lasting relief.

In non-limiting embodiments or aspects, suitable methods include stimulating an area of a patient's central nervous system with a stimulating electrode lead. Those of skill in the art will appreciate that electrode leads (e.g., segmented, non-segmented, unipolar, bipolar, and/or multipolar electrode leads) for such purposes (e.g., for deep brain stimulation (DBS)) are commercially available from, for example, Medtronic Inc. (Minneapolis, Minn. USA), Boston Scientific Corp. (Valencia, Calif. USA), Sapiens Steering Brain Stimulation BV High Tech (AE Eindhoven, The Netherlands), St. Jude Medical, Inc. (now Abbott Laboratories, Austin, Tex. USA), Ad-Tech Medical Instrument Corporation (Racine, Wis. USA, and Integra (Plainsboro, N.J. USA). In addition, systems, including pulse generators and, optionally, electrode leads, are known in the art and are available commercially from, for example, Abbott/St. Jude Medical (including the Infinity DBS and Libra systems), Medtronic (including the Activa and Percept systems), and Boston Scientific (the Vercise and Vercise Gevia systems). In non-limiting embodiments or aspects the system(s) can include an implantable electrode lead, an implantable pulse generator optionally including a computing device and/or having a processor, a communication interface, and/or a storage component (as described below), and rechargeable batteries or non-rechargeable batteries. In non-limiting embodiments or aspects, the electrode lead is a directional electrode lead. In non-limiting embodiments or aspects, only a single electrode lead is implanted for providing stimulation with the parameters as described herein.

In non-limiting embodiments or aspects, the region of the patient's central nervous system that is stimulated, for example by implanting an electrode lead therein, is the basal ganglia. The basal ganglia consists of the caudate, putamen, the globus pallidus (GP), the substantia nigra (including the pars compacta and pars reticulata), and the subthalamic nucleus (STN). In non-limiting embodiments or aspects, any one or more of these anatomical areas are stimulated. In non-limiting embodiments or aspects, any one or more of these anatomical areas are stimulated bilaterally (e.g., the anatomical area in each hemisphere is stimulated), optionally with the same stimulation, optionally each with a different stimulation. In non-limiting embodiments or aspects, the patient's globus pallidus is stimulated, optionally bilaterally. In non-limiting embodiments or aspects, the patient's external globus pallidus (GPe) and/or the patient's internal globus pallidus (GPi) are stimulated, optionally bilaterally. Those of skill in the art will appreciate that various regions of the GP can be mapped using known techniques (e.g., Yelnik et al. Functional mapping of the human globus pallidus: contrasting effect of stimulation in the internal and external pallidum in Parkinson's disease. Neurosci. 2000, 101(1): 77-87), such that an electrode lead, optionally only a single electrode lead can be implanted directly in a region of interest (e.g., the GPe and/or the GPi), optionally bilaterally.

In non-limiting embodiments or aspects, the stimulation that is delivered excites (e.g., causes the formation and/or propagation of an action potential in) parvalbumin (PV)-expressing neurons in the GPe. In non-limiting embodiments or aspects, the stimulation that is delivered inhibits (e.g., prevents the formation and/or propagation of an action potential in) LIM homeobox 6 (Lhx6)-expressing neurons in the GPe. The stimulation that is delivered may both excite PV-expressing neurons in the GPe and inhibit Lhx6-expressing neurons in the GPe. In non-limiting embodiments or aspects, only a single electrode lead is implanted in the GP, optionally in the GPe or GPi, for providing stimulation with the parameters as described herein.

As will be recognized by a person of skill in the art, characteristics of the electrical pulses that are delivered, optionally through only a single implanted electrode lead, optionally through a single lead implanted in each hemisphere of a patient's brain, include, without limitation: intensity (magnitude or size of a signal voltage or current), voltage, amperage, duration, frequency, polarity, phase, pulse-width, relative timing and symmetry of positive and negative pulses in biphasic stimulation, and/or wave shape (e.g., square, sine, triangle, sawtooth, or variations or combinations thereof). Pulse characteristics may be varied in order to provide the desired stimulation and resultant therapeutic effect in a patient or class of patients. So long as other characteristics of the electrical signals (e.g., without limitation, intensity, voltage, amperage, polarity, phase, pulsewidth, relative timing and symmetry of positive and negative pulses in biphasic stimulation, and/or wave shape) are within useful ranges, modulation of the pulse frequency, duration of stimulation, and inter-stimulation interval will achieve the desired therapeutic result(s).

The stimulation that is administered to the patient's central nervous system can include multiple discrete periods, including two or more instances of first periods where a plurality of pulses of electrical stimulation are delivered, optionally through only a single implanted electrode lead, optionally through a single lead implanted in each hemisphere of a patient's brain, and one or more second periods where no pulses of electrical stimulation are delivered (e.g., an inter-stimulation interval). In non-limiting embodiments or aspects, each instance of the two or more instances of first periods has, independently, a duration of from about 50 ms to about 1 second, with the proviso that pulses delivered for about 50 ms are delivered through a single implanted electrode lead, optionally through a single lead implanted in each hemisphere (e.g., not through more than one electrode lead per hemisphere), optionally about 100 to about 400 ms, optionally about 100 to about 300 ms, optionally about 150 to about 250 ms, optionally about 175 to about 250 ms, optionally about 200 to about 250 ms, optionally about 250 ms, and optionally 250 ms, all values and subranges therebetween inclusive. In non-limiting embodiments or aspects, each instance of the one or more instances of second periods has, independently, a duration of from about 500 to about 1900 ms, optionally about 500 ms to about 1000 ms, optionally about 700 to about 900 ms, optionally about 750 to about 850 ms, optionally about 750 to about 825 ms, optionally about 750 to about 800 ms, optionally about 750 ms, optionally 750 ms, all values and subranges therebetween inclusive. In non-limiting embodiments or aspects, each instance of the two or more instances of first periods has a duration of about 250 ms, optionally 250 ms, and each instance of the one or more instances of second periods has a duration of about 750 ms, optionally 750 ms. In non-limiting embodiments or aspects, each instance of the two or more instances of first periods has, independently, a duration of about 100 ms, optionally 100 ms, about 250 ms, optionally 250 ms, about 400 ms, optionally 400 ms, and each instance the one or more instances of second periods has, independently, a duration of about 900 ms, optionally 900 ms, about 1400 ms, optionally 1400 ms, about 1600 ms, optionally 1600 ms, and/or about 1750 ms, optionally 1750 ms. In non-limiting embodiments or aspects, the first periods and the second period have a duration of, respectively, 100 ms and 900 ms, 250 ms and 750 ms, 250 ms and 1750 ms, 400 ms and 1600 ms, and 100 ms and 1400 ms.

In non-limiting embodiments or aspects, the electrical pulses that are delivered, optionally through only a single implanted electrode lead, optionally through a single lead implanted in each hemisphere of a patient's brain, have a pulse width of about 50 to about 500 μs, optionally about 60 to about 150 μs, optionally about 100 μs, and optionally 100 μs, all values and subranges therebetween inclusive.

In non-limiting embodiments or aspects, the electrical pulses that are delivered, optionally through only a single implanted electrode lead, optionally through a single lead implanted in each hemisphere of a patient's brain, have an intensity (amplitude) of about 0.1 mA to about 4 mA, optionally about 1 mA to about 4 mA, all values and subranges therebetween inclusive. In non-limiting embodiments or aspects, the stimulation that is provided is a constant current stimulation. In non-limiting embodiments or aspects, the electrical pulses that are delivered, optionally thorough only a single implanted electrode lead, optionally through a single lead implanted in each hemisphere, have an intensity (voltage) of about 0.1 to about 4 V, all values and subranges therebetween inclusive.

In non-limiting embodiments or aspects, the electrical pulses are delivered, optionally through only a single implanted electrode lead, optionally through only a single lead implanted in each hemisphere, during the two or more first periods at a frequency of from about 100 to about 250 Hz, optionally from about 150 to about 225 Hz, optionally from about 175 to about 200 Hz, optionally about 175 Hz, optionally 175 Hz, optionally about 100 Hz, optionally 100 Hz, all values and subranges therebetween inclusive.

In non-limiting embodiments or aspects, the electrical pulses are delivered through a single implanted electrode lead in or near the GP and associated fiber systems, optionally the GPe or GPi, optionally through a single lead implanted in each hemisphere of a patient's brain, at 175 Hz for a first period of 250 ms, followed by a second period of 750 ms where no pulses are delivered, and this pattern can be repeated one or more times.

It is to be noted that the present methods do not include stimulation at a frequency of between about 100 and about 150 Hz delivered for first periods having a duration of about 50 ms, with second periods where no pulses are delivered having a duration of about 150 ms, when such stimulation is delivered through more than one electrode lead per hemisphere (so-called coordinated resent DBS or CR-DBS).

In non-limiting embodiments or aspects, the electrical pulses that are delivered, optionally through only a single implanted electrode lead, are biphasic pulses. In non-limiting embodiments or aspects, the electrical pulses are charge-balanced pulses.

In non-limiting embodiments or aspects, the first periods are alternated with the second periods for as long as a therapeutic effect results from the stimulation. Optionally, after delivering first periods alternating with second periods for a total duration of 30 minutes to 4 hours, a much longer second period, e.g., at least 1 day, at least 2 days, at least 5 days, at least 1 week, at least 5 weeks, all values and subranges therebetween inclusive, can be included, due to the long-lasting effects of stimulation delivered with the parameters as described herein.

With regard to therapeutic effect, in non-limiting embodiments or aspects, the patient(s) to whom the stimulation as described herein is delivered has/have a movement disorder. In non-limiting embodiments or aspects, the movement disorder is Parkinson's disease, essential tremor, dystonia, and/or Meige Syndrome. In non-limiting embodiments or aspects, the stimulation as described herein, when applied to other areas of the central nervous system known to those of skill in the art to be associated with the condition(s) of interest, can improve one or more symptoms of epilepsy, obsessive-compulsive disorder, depression, addiction, chronic pain, Tourette's Syndrome, and/or obesity. In non-limiting embodiments or aspects, the movement disorder is Parkinson's disease. In non-limiting embodiments or aspects, the movement disorder is Parkinson's disease, and the stimulation described herein improves one or more symptoms thereof, including tremor (e.g., resting tremor), dyskinesia, and/or rigidity in the patient. By “improvise” it is meant that tremor is reduced or eliminated, incidences of dyskinesia are reduced or eliminated, and/or rigidity is lessened or eliminated.

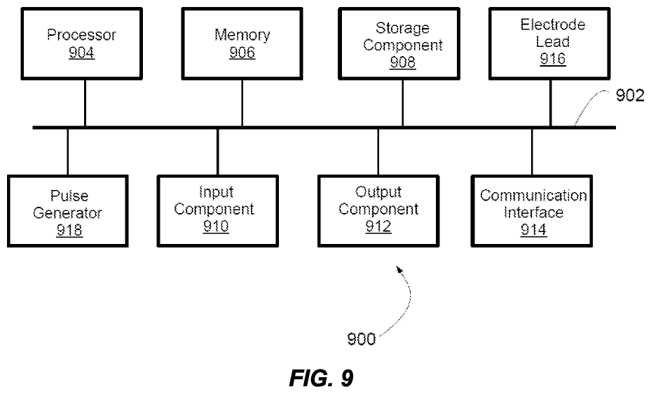

Referring now to FIG. 9, shown is a diagram of example components of a system for providing stimulation with parameters as described herein. As shown in FIG. 9, system 900 may include bus 902, processor 904, memory 906, storage component 908, input component 910, output component 912, communication interface 914, an electrode lead 916 (optionally, only a single electrode lead or two for instances where a single lead is implanted in both hemispheres of a patient's brain), and, optionally, a pulse generator 918. In non-limiting embodiments or aspects, pulse generator 918 is implantable and can be in communication with, through communication interface 914, a non-implantable computing device, such as a desktop computer, laptop computer, smartphone, smart watch, PDA, tablet, etc., which can itself have a communication interface, processor, memory, input component, output component, and/or storage component. Pulse generator 918 can also be in electrical communication with electrode lead 916.

Bus 902 may include a component that permits communication among the components of system 900. In some non-limiting embodiments, processor 904 may be implemented in hardware, software, or a combination of hardware and software. For example, processor 904 may include a processor (e.g., a central processing unit (CPU), a graphics processing unit (GPU), an accelerated processing unit (APU), and/or the like), a microprocessor, a digital signal processor (DSP), and/or any processing component (e.g., a field-programmable gate array (FPGA), an application-specific integrated circuit (ASIC), and/or the like) that can be programmed to perform a function. Memory 906 may include random access memory (RAM), read-only memory (ROM), and/or another type of dynamic or static storage memory (e.g., flash memory, magnetic memory, optical memory, and/or the like) that stores information and/or instructions for use by processor 904.

Storage component 908 may store information and/or software related to the operation and use of system 900. For example, storage component 908 may include a hard disk (e.g., a magnetic disk, an optical disk, a magneto-optic disk, a solid state disk, and/or the like), a compact disc (CD), a digital versatile disc (DVD), a floppy disk, a cartridge, a magnetic tape, and/or another type of computer-readable medium, along with a corresponding drive.

Input component 910 may include a component that permits system 900 to receive information, such as via user input (e.g., a touch screen display, a keyboard, a keypad, a mouse, a button, a switch, a microphone, and/or the like). Additionally or alternatively, input component 910 may include a sensor for sensing information (e.g., a global positioning system (GPS) component, an accelerometer, a gyroscope, an actuator, and/or the like). Output component 912 may include a component that provides output information from system 900 (e.g., a display, a speaker, one or more light-emitting diodes (LEDs), and/or the like).

Communication interface 914 may include a transceiver-like component (e.g., a transceiver, a separate receiver and transmitter, etc.) that enables device to communicate with other devices (including, e.g., a pulse generator, such as an implantable pulse generator), such as via a wired connection, a wireless connection, or a combination of wired and wireless connections. Communication interface 914 may permit system 900 to receive information from another device (such as a computing device) and/or provide information to another device (such as a computing device). For example, communication interface 914 may include an Ethernet interface, an optical interface, a coaxial interface, an infrared interface, a radio frequency (RF) interface, a universal serial bus (USB) interface, a Wi-Fi® interface, a cellular network interface, BLUETOOTH interface, and/or the like. Suitable communication protocols and methods for securing communications between communication interface 914 of an implantable system 900 and a communication interface of another device, such as a computing device (e.g., desktop computer, laptop computer, smartphone, smart watch, PDA, tablet, etc.) can include encryption, e.g., using a secure socket layer (SSL) (e.g., by using public/private key pairs as are known in the art). Additional security protocols are disclosed in, for example, U.S. Pat. Nos. 9,445,264 and 9,463,325, the contents of which are hereby incorporated by reference in their entirety.

System 900 may perform one or more stimulation processes described herein. System 900 may perform these processes based on processor 904 executing software instructions stored by a computer-readable medium, such as memory 906 and/or storage component 908, and/or being instructed by a separate computing device. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 906 and/or storage component 908 from another computer-readable medium or from another device via communication interface 914. When executed, software instructions stored in memory 906 and/or storage component 908 may cause processor 904 to perform one or more processes described herein, for example, causing pulse generator 918 to deliver electrical stimulation through electrode lead 916 with the parameters described herein. Additionally or alternatively, hardwired circuitry may be used in place of or in combination with software instructions to perform one or more processes described herein. Thus, embodiments described herein are not limited to any specific combination of hardware circuitry and software.

The number and arrangement of components shown in FIG. 9 are provided as an example. For example, an implantable system 900 can include electrode lead 916 in electrical communication with a pulse generator device, optionally an implantable pulse generator device, which can include bus 902, processor 904, memory 906, storage component 908, input component 910, output component 912, and/or communication interface 914, together with a pulse generator 918. In non-limiting embodiments or aspects, an implantable pulse generator device can include bus 902, processor 904, memory 906, communication interface 914, and pulse generator 918. A useful system can include one or more of any component identified in FIG. 9, in some non-limiting embodiments or aspects, system 900 may include additional components, fewer components, different components, or differently arranged components than those shown in FIG. 9. For example, an external (e.g., not implanted) portion of a system as described herein can include a computing device and/or a processor, memory, storage component, input component, output component, and communication interface, while an implantable portion of system can include an electrode lead, a pulse generator, and one or more other components such as a computing device and/or processor, memory, storage component, and communication interface for, for example, communicating with the external component of the system. Additionally or alternatively, a set of components (e.g., one or more components) of system 900 may perform one or more functions described as being performed by another set of components of system 900.

### Example 1

An investigation of basal ganglia circuit architecture was undertaken using electrical stimulation as a probing device, and the results were then used to devise a form of cell-type specific, electrically-driven, neuromodulation. In addition, a broad parameter space was explored with a machine learning approach to identify parameters that maximize the cellular specificity of stimulation.

The experiments described hereinafter identified parameters that emulate the same pattern of cell-type specific responses induced using optogenetics—preferentially exciting PV-GPe neurons while simultaneously inhibiting Lhx6-GPe neurons—using a single point of electrical stimulation.

The experiments further identified the circuit elements required to drive these cell-type specific responses. These results establish the feasibility of using electrical stimulation to drive differential responses by cell-type in the central nervous system and suggest the potential for developing a more robust toolbox for deep brain stimulation therapies. These rapidly translatable stimulation protocols can immediately be tested in PD models across species and have the added benefit of falling within current FDA approved frequency ranges for human use.

### Materials and Methods

### Animals

Experimental procedures were approved by the Carnegie Mellon University Committee for the Use and Care of Animals and in accordance to the guidelines set forth by the National Institute of Health and Society for Neuroscience Use of Animals in Neuroscience Research. Male and female heterozygous mice 4-15 weeks old on a C57BL/6J background were used for all experiments. PV-GPe neurons and Lhx6-GPe neurons were targeted using double transgenic mice from a PV-tdTmt+/−; Lhx6GFP+/− mouse line. D1 neurons were targeted using a D1-Cre+/−; PV-tdTmt+/−; Lhx6GFP+/− mouse line. Striatopallidal neurons were targeted using an A2ACre+/−; PV-tdTmt+/−; Lhx6GFP+/− mouse line. Animals were group housed (2-8 per group) in a 12-h/12-h light dark cycle until used for experimentation.

### Surgical Procedures and Viral Transfection

Injections of purified double-floxed AAV2-syn-FLEX-Chronos-GFP (cell-specific activation) or AAV2-syn-Chronos-GFP (pan-neuronal activation) produced at the University of North Carolina (Vector Core Facility) were made in D1-Cre+/− (JAX: 024860); PV-tdTmt+/− (JAX: 027395); Lhx6GFP+/− (MMRC 000246-MU), or A2A-Cre+/− (JAX: 010687); PV-tdTmt+/−; Lhx6GFP+/−, transgenic mice 4-12 weeks old. Injections were made into the subthalamic nucleus by methods previously described. Briefly, anesthesia was induced using ketamine (100 mg/kg) and xylazine (30 mg/kg) and maintained throughout surgery using 1.5% isoflurane. Mice were placed in a stereotaxic frame (Kopf Instruments), where the scalp was opened and bilateral holes were drilled in the skull (striatum, 2 sites: 1.35 mm anterior, ±1.9 mm lateral; 0.95 mm anterior, ±1.4 mm lateral. STN: −1.45 mm anterior, ±1.52 mm lateral from bregma). Virus (STN: 60 nL; Striatum: 1.75 μL, 1.25 μL) was injected with a Nanoject (Drummond Scientific) through a pulled glass pipet (tip diameter ˜30 μm) whose tip was positioned below the top of the skull (striatum: 2.75 mm and 2.5 mm, STN: 4.5 mm) To prevent backflow of virus, the pipet was left in the brain for 5-8 min after completion of each injection. All experiments were performed at least 4 weeks after injection to allow time for full viral expression.

### Quantification and Statistical Analysis

For each analysis describe throughout, values were measured from average responses of 5 consecutive sweeps and data are expressed as mean±standard deviation in the text unless otherwise indicated. Figure legends indicate statistical measures represented in figures. All statistical analyses described herein were performed in IBM SPSS, version 24.

For each cell, baseline firing rate was calculated as the average firing rate over the 2 s prior to stimulation, and during stimulation, per trial. Stimulus firing rate was conducted after stimulation artifact removal; 0.07-0.11 ms were removed for each stimulus to subtract electrical stimulation artifacts (no subtraction necessary for optogenetic experiments). Action potentials that occurred during the stimulus window were then counted (per trial) and divided by the stimulation duration to calculate the stimulus firing rate. A modulation factor (MF) was then calculated for each trial: MF=(FRₛₜᵢₘ−FR_{baseline})/(FRₛₜᵢₘ+FR_{baseline}), and these modulation factors per trial were averaged to calculate a mean modulation factor per neuron, which is plotted as a single point in figures. Paired t tests of baseline firing rate vs. firing rate during stimulation were used to determine if a neuron was significantly modified from its baseline over multiple trials. Any neurons that were not significantly modified are noted with gray points throughout figures. Comparisons between cell-types for any given condition were made with a Mann Whitney U (Wilcoxon's nonparametric).

Statistical analysis regarding EPSCs, IPSCs, and inhibition-excitation balance was performed using Kruskal-Wallis analysis of variance (ANOVA) nonparametric test (KW) and any differences were further investigated with a Wilcoxon nonparametric (Mann-Whitney U) pairwise comparison between cell types with a Bonferroni correction. Statistical analysis of 100 hz inhibitory charge decay was performed using a Wilcoxon's nonparametric test (Mann Whitney U).

### Electrophysiological Recordings

Parasagittal sections (300 μm thickness) containing the GPe were prepared from brains of 5- to 14-week-old mice. Slices were prepared on a Leica VT1200 vibratome in an ice-cold HEPES cutting solution (in mm) 92 NaCl, 2.5 KCl, 1.2 NaH2PO4, 30 NaHCO3, 20 HEPES, 25 glucose, 5 sodium ascorbate, 2 thiourea, 3 sodium pyruvate, 10 MgCl2, and 0.5 CaCl2. Slices were allowed to recover for 15 min at 33° C. in a chamber filled with N-methyl-d-glucamine (NMDG)-HEPES recovery solution (in mm) 93 NMDG, 2.5 KCl, 1.2 NaH2PO4, 30 NaHCO3, 20 HEPES, 25 glucose, 10 MgSO4, 0.5 CaCl2, 5 sodium ascorbate, 2 thiourea, and 3 sodium pyruvate. After 15 min, slices were held at room temperature for at least 1 h before recording in a holding solution that was similar to the HEPES cutting solution but with 1 mm MgCl2 and 2 mm CaCl2. Recordings were made at 33° C. in carbogenated ACSF (in mm) 125 NaCl, 26 NaHCO3, 1.25 NaH2PO4, 2.5 KCl, 12.5 glucose, 1 MgCl2, and 2 CaCl2.

Data were collected with a MultiClamp 700B amplifier (Molecular Devices) and ITC-18 analog-to-digital board (HEKA) using Igor Pro software (Wavemetrics) and custom acquisition routines (Recording Artist; Richard C. Gerkin, Phoenix, Ariz.). Voltage-clamp recordings were filtered at 2 kHz and digitized at 10 kHz. Recording electrodes were made from borosilicate glass (pipette resistance, 2-4 MΩ) (World Precision Instruments). Different internal solutions were used depending on the experiment. The internal solution for all extracellular recordings consisted of the following (in mM): 130 KMeSO3, 10 NaCl, 2 MgCl2, 0.16CaCl2, 0.5 EGTA, 10 HEPES, 2 Mg-ATP, 0.3 Na-GTP; pH 7.5. The internal solution for intracellular recordings consisted of the following (in mM): 120 CsMeSO3, 0.5 EGTA, 10 BAPTA, 10 HEPES, 2 Mg-TP, 0.3 Na-GTP, 5 QX-314; pH 7.3. This low chloride internal resulted in ECl-(−85 mV). Electrical stimulation was delivered through a stimulus isolator (ISO-Flex) connected to a concentric bipolar electrode (FHC CBBPF75) at described stimulation amplitudes. The stimulating electrode was placed in the internal capsule fibers between the GPi and the GPe.

Extracellular recordings: For measurements of autonomous or driven firing in neurons, cell-attached recordings were used to prevent disruption of intracellular milieu. Recordings were made 2-3 minutes after securing a 20-75 MΩ seal in spontaneously firing neurons.

Intracellular recordings: For whole-cell voltage clamp recordings, cells were first clamped at −70 mV after break-in and allowed to recover for 3-5 minutes. Rs was monitored throughout the experiment, and cells with an Rs over 40 MΩ, or cells where Rs changed by more than 30% over the course of the recording were excluded. Measurements of peak EPSC amplitude within 20 ms of stimulation were then made, and decay constants was calculated on the first event in any given stimulus series. To measure inhibitory currents, neurons were then ramped from −70 mV to 0 mV over 2 s. Peak IPSC amplitude and decay constants were calculated as described above. All synaptic response amplitudes were calculated by finding the average amplitude of evoked PSCs in five consecutive trials with an intertrial interval of 20 s unless noted otherwise.

Electrical stimulation: While extracellularly attached, electrical stimulation trains at described frequencies, durations, and stimulation amplitudes/intensities were delivered to sequentially recorded cells within 100 μm of each other. Frequency, duration, and stimulus intensity order were identical for each neuron in an in-field pair, but pseudo-randomly varied between pairings. Pulse widths of 0.1 ms were used throughout all electrical stimulation experiments.

Optogenetic stimulation: Optical stimulation was delivered at 100 Hz for 1 s with a pulsewidth of 1 ms for all optogenetic experiments (filtered at 470 nm, delivered through the 60× objective of the rig microscope). Due to the use of a PV-tdTmt+/−; Lhx6GFP+/− mouse line and GFP-tagged Chronos in optogenetic experiments, the presence of fluorescence in a field was insufficient to indicate successful viral transfection. A neuron in a given field was tested with 0.05 mW light power. If there was no response, laser power was incrementally raised up to maximum power (1 mW), stopping when a response was observed (average across responding cells ˜0.5 mW). An in-field partner neuron would then be tested at the same laser power. Pairs where neither neuron responded to 1 mW were excluded as this could be due to insufficient virus expression in the region.

Drugs: NBQX disodium salt and D-APV were obtained from Tocris Bioscience. Each drug was diluted to the appropriate concentration by adding to the superfusate at the beginning of each experiment.

### Immunohistochemistry

Imaging of striatonigral and striatopallidal projections was completed through enhancing and imaging AAV2-syn-FLEX-Chronos-GFP which had been injected in D1-Cre+/− and A2A-Cre+/−; PV-tdTmt+/− transgenic mice 4-12 weeks old. Brains were fixed in 4% PFA for approximately 24 hours before being moved to 30% sucrose. GFP was enhanced with chicken anti-GFP (1: 1000, Ayes Laboratory) incubated for 24 h at room temperature. Sections were then incubated in AlexaFluor-488 anti-chicken (1:500, Invitrogen) for 1.5 h at room temperature. Tissue was then mounted and cover-slipped with gelvatol prior to imaging.

### Computational Methods

Putative optimal parameter values were identified by training a machine learning model to use the log of the duration, amplitude, and frequency to predict the MFs of each cell type, evaluating the machine learning models' predictions for different values of these parameters, and selecting parameter values for which the model predicted a high population separation index (PSI) between PV and Lhx6 neurons. Data was collected by presenting 19 pairs of PVGPe and Lhx6-GPe neurons with approximately 10 different burst parameter combinations. Burst combinations were randomly generated from the parameters in Table 1 in Excel.

TABLE 1
Parameters Included in Iteration 1

| | |
|---|---|
| Frequencies (Hz) | 10, 20, 50, 80, 100, 150, 200 |
| Duration (ms) | 50, 100, 250, 500, 1000, 5000, 10000, 20000, |
| | 30000 |
| Threshold Multipliers | 0.5, 1.0, 1.25, 1.5, 2.0, 2.5 |

For 9 of the pairs, a new randomly generated set of parameter combinations was used for each neuron. For the remaining 10 pairs, the same set of parameter combinations was used for each neuron in the pair. In any experiment, recording was stopped if a cell's baseline firing rate dropped to 0 Hz. As generation of parameter combinations was randomized, there was overlap for some combinations in the data set, resulting in 208 unique combination. A Gaussian process regression with a quadratic polynomial kernel was trained, allowing for putative non-linear relationships between parameters, and evaluated the leave-one-out cross-validation performance, where the held-out “example” was all of the samples for a single parameter values combination. In the leave-one-out cross-validation, the models were trained on the raw modulation indices, and the correlation coefficients were calculated between the model's prediction on each set of unique parameters and that parameter set's average modulation index.

Gaussian process regression models were fit using the R kernlab package. Spearman and Pearson correlation coefficients were calculated between the predicted MFs and the average MFs across each parameter value combination. Unfortunately, sufficient data was not generated to have a separate held-out test set to evaluate how well the model generalizes to examples that were not used for training or model selection.

The selected models were then used to identify parameter values that would be likely to improve PSI. This was done by making predictions on thousands of artificially generated parameter value combinations for each model and identifying combinations that led to large predicted PSI (Table 2).

TABLE 2
Sampling Rates to Generate Model Predictions

| | |
|---|---|
| Frequencies (Hz) | Every 5 Hz between 0 Hz and 200 Hz |
| Duration (ms) | Every 20 ms between 20-2500 ms; every |
| | 1000 ms between 3000 ms and 30000 ms |
| Amplitude Multipliers | Every 0.05 between 0.5× and 2.5× |

The predicted PSI was computed by subtracting the predicted MFLhx6 from the predicted MFPV. After obtaining these results, more experiments were performed to explore values around the predicted optimal parameter values to determine if such values would produce greater PSIs than the parameter values that had been previously tested. The above process of training two Gaussian process regression models was repeated for the newly acquired data in addition to the previously acquired data. Each model was then used again to make predictions on the parameter values from Table 2 and computed the predicted PSI. More experiments were performed for values close new these new predicted optimal parameter values as well as parameter values that were far from these optimal parameter values.

### Results

### PV-GPe and Lhx6-GPe Neurons Respond Similarly to Conventional High Frequency Electrical Stimulation

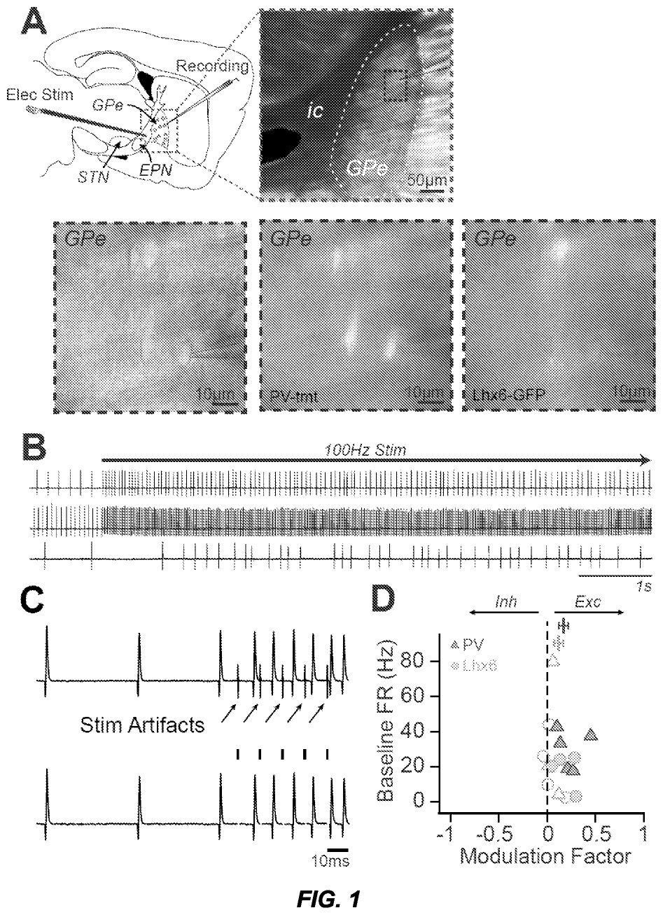

To investigate the neuromodulatory effects of DBS on neuronal subpopulations in the GPe, PV-GPe and Lhx6-GPe neurons in parasagittal brain slices of Lhx6-GFP+/−; PVtdTmt+/− mice were targeted (FIG. 1, panel A). Firing rates were monitored with cell-attached recordings during electrical stimulation of the internal capsule, containing the axon bundle from the subthalamic nucleus (STN) to the GPe (100 Hz, 30 s, 5 trials per neuron) (FIG. 1, panel B, representative trials from 3 different neurons). Recordings were made from pairs of PV-GPe and Lhx6-GPe neurons, recorded sequentially in pseudorandom order within the same field of view (<50 μm apart). Stimulation artifacts were digitally subtracted offline, allowing for examination of firing rate modulation during stimulation (FIG. 1, panel C).

Stimulation generally resulted in a modest increase in firing rates over baseline, quantified by computing a modulation factor for each neuron: (FRₛₜᵢₘ−FR_{baseline})/(FRₛₜᵢₘ+FR_{baseline}). FIG. 1, panel D shows the results of recordings from 8 pairs of neurons, across N=3 animals, in which at least one neuron showed significant firing rate modulation during stimulation (p<0.05, paired t-test across 5 trials, shaded symbols). Neurons which were not significantly modulated during stimulation are indicated in with open symbols, but their responses were still included in calculations of population-level responses. The average modulation factor across all Lhx6-GPe neurons was 0.12±0.13 and across all PV-GPe neurons was 0.17±0.14 (p=0.42 Mann-Whitney U), and no consistent relationships were seen between baseline firing rates and the degree of modulation during stimulation (FIG. 1, panel D). These results suggest that continuous high frequency electrical stimulation, as used during conventional DBS, drives significant but small increases in the firing rates of both PV-GPe and Lhx6-GPe neurons, consistent with previous reports (Moran et al., Dynamic stereotypic responses of basal ganglia neurons to subthalamic nucleus high-frequency stimulation in the parkinsonian primate. Frotn Syst. Neurosci. 2011, 5: 21; So et al., Frequency-dependent, transient effects of subthalamic nucleus deep brain stimulation on methamphetamine-induced circling and neuronal activity in the hemiparkinsonian rat. Behav. Brain. Res. 2017, 320: 119-127). In FIG. 1, panel D, neurons in which stimulation drove significant changes in firing rate (p<0.05, paired t-test FR_{baseline} vs. FRₛₜᵢₘ, Trials 1-5) are shaded darker, while neurons in which firing rate changes were not significant are not shaded. Horizontal markers show average MFLhx6 and MFPV, error bars: SEM, calculated across all neurons, regardless of whether FR changes were significant (n=8 pairs of neurons; N=3 mice).

I-E Ratio is Higher onto Lhx6-GPe Neurons Compared to PV-GPe Neurons

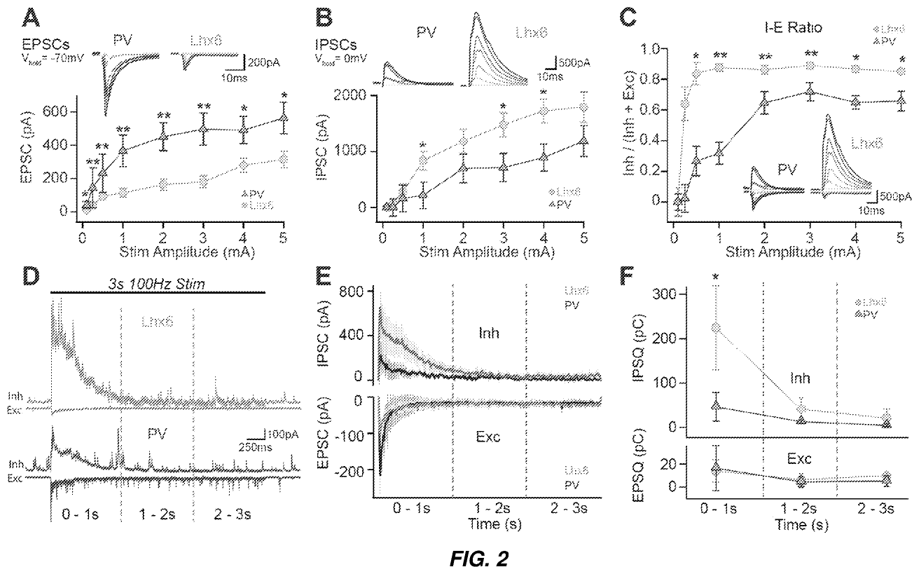

To study the synaptic inputs recruited onto GPe neurons during stimulation, whole-cell voltage clamp recordings were performed. Stimulus-response curves were constructed for excitatory postsynaptic currents (EPSCs), measured from a holding potential of −70 mV, and for inhibitory postsynaptic currents (IPSCs), measured from a holding potential of 0 mV in the same neurons (FIG. 2, panels A and B). Data were collected from pairs of PV-GPe and Lhx6-GPe neurons, patched sequentially in pseudorandom order, <50 μm apart. As shown in FIG. 2, panel A, EPSCs recorded in PV-GPe neurons were larger than those recorded in Lhx6-GPe neurons across stimulation intensities (KW, χ2(7)=44.4, p<0.00001). The decay kinetics of EPSCs were similar between both cell types (τPV=3.6±0.9 ms; τLhx6=4.8±4.2 ms; Mann Whitney U, p=0.74, n=14 pairs across 7 mice). Conversely, IPSCs recorded in Lhx6-GPe neurons were larger than those recorded in PV-GPe neurons (KW, χ2(7)=8.45, p=0.004) (FIG. 2, panel B). There was no significant difference in the decay kinetics of IPSCs between cell types (τPV=11.6±8.5 ms; τLhx6=13.6±10.3 ms; Mann Whitney U, p=0.654).

FIG. 2, panel C shows the average inhibition-excitation (I-E) ratio [(IPSO)/(IPSC+EPSC)] recorded in PV-GPe and Lhx6-GPe neurons at each stimulus intensity. A score of 0.5 indicates that amplitudes of excitatory and inhibitory synaptic currents were equally balanced. The I-E ratio in Lhx6-GPe neurons was consistently >0.5, meaning that inhibition dominated at all but the lowest stimulus intensities (FIG. 2, panel C). In contrast, in PV-GPe neurons, excitation FIG. 2, panel C). These results establish that PV-GPe and Lhx6-GPe populations experience different balances of synaptic inhibition and excitation during stimulation (KW, χ2(7)=57.4, p<0.00001), and the magnitude of population-specific differences varies as a function of stimulus intensity (FIG. 1, panel D).

Cell-specific differences were most pronounced for stimulus amplitudes between 0.5-1.5 mA. To investigate the dynamics of synaptic inputs during continuous stimulation, neurons were challenged with 100 Hz stimulus trains (1 mA), often used in DBS. Once again, recordings were performed in paired PV-GPe and Lhx6-GPe neurons. Stimulation at 100 Hz drove pronounced and rapid synaptic depression of both EPSCs and IPSCs (FIG. 2, panels D and E). By the end of a 3 s stimulus, EPSCs were depressed to 14.3±17.8% of their initial response in PV-GPe neurons and to 25.7±13.7% in Lhx6-GPe neurons (Mann Whitney U, p=0.035, n=10 pairs across 3 mice). Despite the larger amplitude of EPSCs in PV-GPe neurons initially, there was no significant difference in the overall charge transferred through excitatory conductances between PV-GPe and Lhx6-GPe neurons across the 3 s stimulus (EPSQ_{PV}=−54.7±89.8 pC, EPSQ_{Lhx6}=−55.9±55.0 pC, Mann Whitney U, p=0.739) (FIG. 2, panel F). Stimulation at 100 Hz also drove marked depression of IPSCs: to 9.5±10.9% in PV-GPe neurons and to 13.8±10.5% in Lhx6-GPe neurons (Mann Whitney U, p=0.278) (FIG. 2, panels D and E).

Over the 3 s stimulus, significantly more charge was transferred through inhibitory conductances in Lhx6-GPe neurons compared to PV-GPe neurons (IPSQLhx6=392.5±444.7 pC, IPSQPV=123.5±114.3 pC, Mann Whitney U, p=0.009). This difference was most pronounced within the first 1 s of stimulation (IPSQ_{Lhx6}=284±300 pC, IPSQ_{PV}=98±103 pC, Mann Whitney U, p=0.023) (FIG. 2, panel F).

Taken together, these results suggest that during high frequency stimulation, as used in DBS, a timing window exists where Lhx6-GPe neurons experience more synaptic inhibition than PV-GPe neurons; the boundaries of this window are defined by the biophysical properties of synaptic depression.

### Short Bursts of Electrical Stimulation Segregate Responses of PV-GPe and Lhx6-GPe Neurons

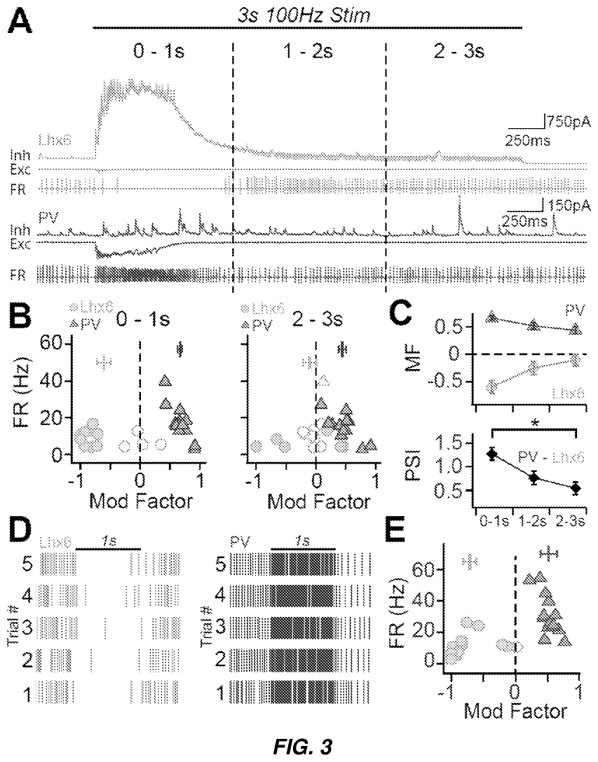

Results from the synaptic experiments identify stimulus features that differentiate the synaptic drives onto PV-GPe and Lhx6-GPe neurons during electrical stimulation. To test whether these synaptic differences are sufficient to differentiate the firing responses of PV-GPe and Lhx6-GPe neurons during stimulation, the correlation between synaptic currents and extracellular firing rates was explored. Sequential extracellular/intracellular recordings were performed from a subset of PV-GPe and Lhx6-GPe neurons (FIG. 3, panel A). Neurons were first patched in cell-attached configuration to measure their firing responses to stimulation. Next, the same neurons were re-patched in whole-cell voltage clamp configuration to measure their underlying synaptic currents evoked during stimulation. Excitatory and inhibitory currents were measured in each neuron by setting the holding potential to −70 mV or 0 mV, respectively. FIG. 3, panel A shows recordings from two representative neurons, in which the large inhibitory current in Lhx6-GPe neurons was well correlated with a transient suppression of extracellular firing rate. In contrast, the PV-GPe neuron, which lacked a large inhibitory current, was instantly excited by stimulation.

Extracellular recordings across a wider sample of Lhx6-GPe and PV-GPe neurons revealed that Lhx6-GPe were routinely inhibited during the first 1 s of 1 mA stimulation, but less so in subsequent time bins (FIG. 3, panels B and C). In contrast, PV-GPe neurons were often excited throughout, with only a modest decrease over time (FIG. 3, panels B and C). Firing rate modulation during stimulation was quantified by computing each neurons' modulation factor (MF) over each time bin of stimulation (0-1 s, 1-2 s, 2-3 s) (FIG. 3, panel C) No relationship was seen between a neurons' baseline firing rate and its degree of modulation (FIG. 3, panel B). Across all time bins, PV-GPe neurons exhibited increased firing rates, indicated by positive MFs, and this effect decreased slightly but significantly over time (KW, χ2(2)=9.6, p=0.008). In contrast, Lhx6-GPe neurons were initially strongly inhibited but became less inhibited over time, as indicated by a strong negative MF in the 0-1 s time bin that became less negative over time (KW, χ2(2)=7.9, p=0.019) (FIG. 3, panel C), with some neurons even switching from being inhibited at the beginning of stimulation to being excited by the end (FIG. 3, panel B). To quantify the degree of separation between the firing responses of Lhx6-GPe and PV-GPe neurons, a ‘population separation index’ (PSI) for each pair (PSI=MFPV−MFLhx6) was computed. On average, the PSI between pairs of Lhx6-GPe and PV-GPe neurons was 1.27±0.49 in the first 1 s time bin, but only 0.77±0.54 in the second time bin, and 0.55±0.49 in the third time bin (KW, χ2(2)=12.0, p=0.003) (FIG. 3, panel C). These results suggested that delivering stimulation in bursts (˜1 s in duration), rather than continuously, would drive opposite firing responses in PV-GPe and Lhx6-GPe neurons. To test this hypothesis, repeated bursts (1 s duration, 100 Hz, 1 mA) were delivered to pairs of Lhx6-GPe and PV-GPe neurons. As shown in FIG. 3, panels D and E, this drove robust and reproducible suppression of Lhx6-GPe neurons (MFLhx6=−0.7±0.3), and excitation of PV-GPe neurons (MFPV=0.5±0.1) (PSI=1.2±0.4, FIG. 3, panels D and E).

### STN Inputs Excite Both PV-GPe and Lhx6-GPe Neurons

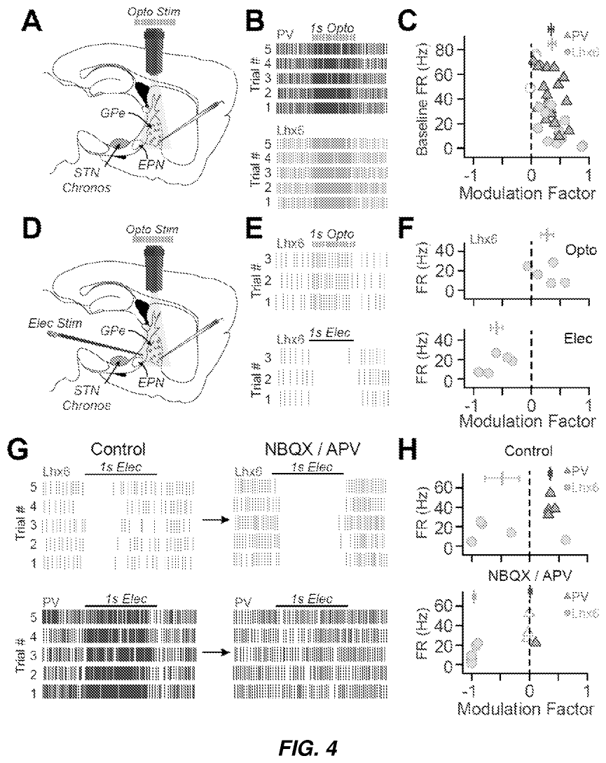

Given the critical role of inhibitory synaptic transmission in differentiating the responses of PV-GPe and Lhx6-GPe neurons with electrical stimulation, identification of the mechanism underlying this cell-type specificity was attempted. One hypothesis is that excitatory drive from the STN engages the GPe's inhibitory collateral network. To test whether the observed synaptic inhibition reflected the feedforward activity of GPe collaterals, tests were run to determine whether the same pattern of cell-type specific inhibition could be reproduced by optogenetically stimulating the STN. STN neurons were virally transfected with Chronos, a fast firing variant of channelrhodopsin (AAV2-syn-Chronos-ChR2-GFP). Experiments were performed in Lhx6-GFP+/−; PV-tdTmt+/− mice to differentiate these populations during recordings. Parasagittal slices were prepared 4-8 weeks after injections. Firing rates were recorded in cell-attached configuration from pairs of PV-GPe and Lhx6-GPe neurons during optical stimulation of STN fibers in the GPe (1 s, 100 Hz, ˜0.5 mW) (FIG. 4, panel A). Data are included from pairs in which at least one neuron responded to stimulation. Optogenetic activation of STN fibers resulted in excitation of both PV-GPe and Lhx6-GPe neurons (MFLhx6=0.36±0.27, MFPV=0.34±0.17; p=0.976, Mann Whitney U; n=17 pairs of neurons across 3 animals) (FIG. 4, panels B and C), arguing against a feedforward mechanism for inhibition.

To confirm that the same Lhx6-GPe neurons excited by STN stimulation were inhibited by electrical stimulation, neurons were tested with both optical and electrical stimulation trials (FIG. 4, panel D). For these experiments, Lhx6-GPe neurons were targeted for cell-attached recordings and presented with three optical stimulation trials (100 Hz, 1 s, —0.5 mW) and three electrical stimulation trials (100 Hz, 1 s, 1 mA). As shown in FIG. 4, panels E and F, the same individual neurons that were excited by optogenetic stimulation of the STN were inhibited by electrical stimulation (MF_{Opto}=0.27±0.25; MF_{Elec}=−0.6±0.23) (n=5 neurons across 2 mice). To rule out the possibility that excitation from any other source (i.e. cortex) might recruit feedforward inhibition in the GPe, inhibitory responses during electrical stimulation were tested to determined whether they could be blocked by bath application of the glutamate receptor blockers, 10 μM NBQX and 50 μM D-APV. Neurons were not paired for this experiment because bath application of drugs precluded recording from more than one neuron per slice. As shown in FIG. 4, panels G and H, application of NBQX/APV blocked excitation of PV-GPe neurons during stimulation (MF_{Ctrl}: 0.36±0.05, MF_{NBQX/APV}: 0.01±0.07, p=0.0001 paired t test), but did not block inhibition of Lhx6-GPe neurons (MF_{Ctrl}: −0.47±0.66, MF_{NBQX/APV}: −0.96±0.05, p=0.18, paired t test).

Taken together, these results suggest that feedforward inhibition is not the mechanism responsible for the preferential inhibition of Lhx6-GPe neurons seen during electrical stimulation.

### Collaterals of Striatonigral Neurons Preferentially Inhibit Lhx6-GPe Neurons

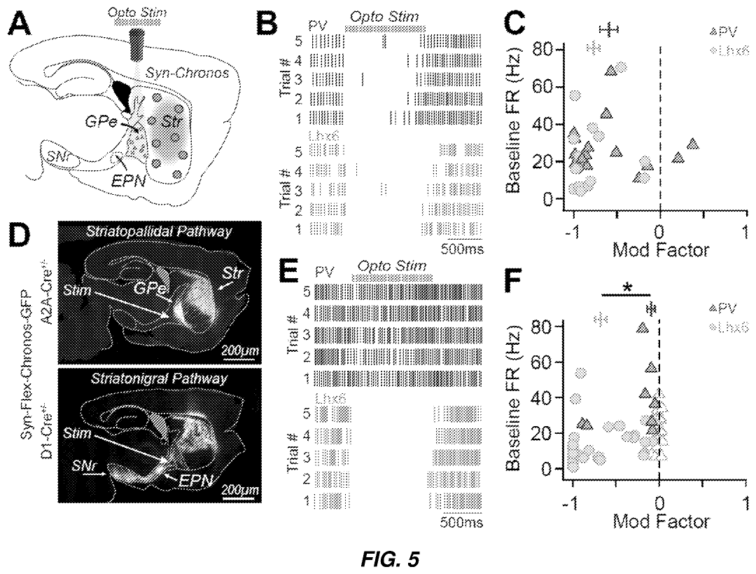

The next hypothesis was that inhibition of Lhx6-GPe neurons was driven by the activation of direct inhibitory inputs to the GPe. The striatum is the major source of inhibitory input to the GPe. Striatopallidal projections have been shown to broadly innervate most cell types in the GPe, including PV-GPe neurons, but the magnitude of their effects on the firing rates of PV-GPe vs. Lhx6-GPe neurons has not been explicitly measured. To test the functional effects of striatal inhibition on PV-GPe vs. Lhx6-GPe neurons, pan-neuronal Chronos (AAV2-syn-Chronos-ChR2-GFP) was injected into the striatum of 5-10-week old Lhx6-GFP+/−; PV-tdTmt+/− mice (FIG. 5, panel C). Parasagittal slices were prepared 4-8 weeks after injections and cell-attached recordings were performed to monitor the firing rates of PV-GPe and Lhx6-GPe neurons during optogenetic stimulation (1 s, 100 Hz, —0.5 mW) of striatal fibers in the GPe (FIG. 5, panel A). Data are included from pairs where at least one neuron responded to stimulation. Optogenetic activation of striatal fibers in the GPe drove inhibition in both PV-GPe and Lhx6-GPe neurons (MF_{Lhx6}=−0.77±0.29, MF_{PV}: −0.59±0.41; p=0.25 Mann Whitney U; n=16 pairs of neurons across 3 animals) (FIG. 5, panels B and C).

At face value, these results seemed to suggest that projections from the striatum lack the cell-type specificity needed to selectively inhibit Lhx6-GPe neurons. But considering the placement of the stimulating electrode, the electrical experiments are unlikely to be activating this canonical striatopallidal pathway (FIG. 5, panel D, top). Instead, the stimulating electrode is much closer to axons of striatonigral neurons, projecting from the striatum to the substantia nigra reticulata (SNr) and internal globus pallidus (GPi), but which also extend inhibitory collaterals to the GPe (FIG. 5, panel D, at bottom). To selectively stimulate the striatonigral pathway, Chronos was selectively expressed in dopamine D1-type receptor-expressing spiny projection neurons (D1-SPNs) by injecting AAV2-DIO-Chronos-GFP into the striatum of D1-Cre+/−; PV-tdTmt+/−; Lhx6GFP+/− triple transgenic mice (FIG. 5, panel D, bottom). In response to optical stimulation of D1-SPN fibers in the GPe, Lhx6-GPe neurons were robustly inhibited (MF_{Lhx6}=−0.681±0.34), but PV-GPe neurons were not (MF_{PV}=−0.099±0.23; p<0.00001 Mann Whitney U; n=27 pairs across 4 mice) (FIG. 5, panels E and F).

These results reveal a previously unknown cell-type selectivity in the distribution of striatopallidal inputs onto PV-GPe and Lhx6-GPe populations, and a mechanism through which electrical stimulation can drive selective inhibition of Lhx6-GPe neurons.

Taken together, the combined results of the optogenetic experiments (FIGS. 4 and 5) suggest that bimodal responses evoked in PV-GPe and Lhx6-GPe populations during electrical stimulation reflect convergent effects of excitatory inputs from the STN and inhibitory inputs from striatonigral neurons.

### Burst Parameter Optimization for Cell-Type Specificity Using a Machine Learning Approach

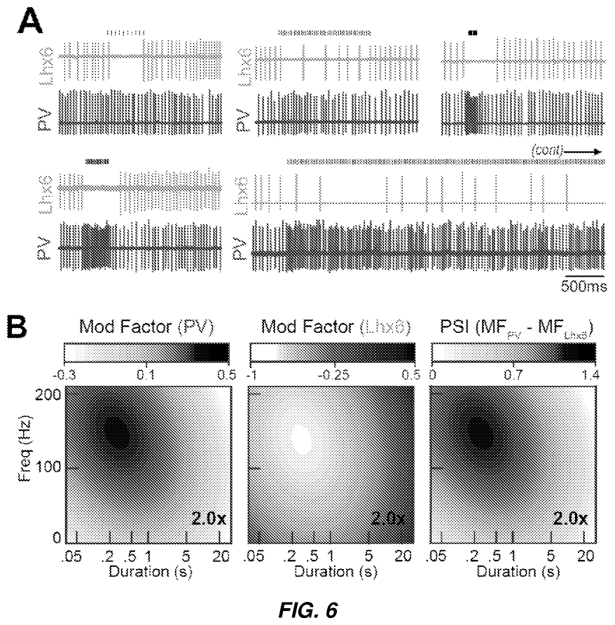

To determine how stimulation parameters can be tuned to maximize separation in the responses of PV-GPe and Lhx6-GPe populations, an active machine learning approach, in which experimental data was used to iteratively train models that could predict the nonlinear relationships between combinations of stimulus parameters and firing rates of PV-GPe and Lhx6-GPe populations, was used. To generate training data sets for these models, PV-GPe and Lhx6-GPe neuron pairs were presented with approximately ten different burst combinations, with parameters randomly assigned from the values shown in Table 1 (FIG. 6, panel A, sample responses).

Instead of using concrete stimulation amplitudes (i.e. 1 mA), a thresholded value (i.e. 2×) was used to account for slice-to-slice variability in electrode placement and overall tissue excitability. ‘Threshold’ was calculated empirically for each slice by averaging the minimum stimulation intensity required to drive significant changes in firing rates from baseline, measured in three different neurons using a 1 s, 100 Hz burst. The average intensity across the three cells was the ‘threshold’ value for the slice, ranging from 0.05-0.83 mA for these experiments.

After gathering a data set of 208 unique burst combinations collected across 19 pairs of Lhx6-GPe and PV-GPe neurons, the data was used to train Gaussian process regressions with quadratic polynomial kernels for each population; this approach was selected because it incorporates non-linear relationships between the features and dependent variables as well as pairwise interactions between the features. Performance of the models across thousands of artificially generated burst combinations (Table 2) was assessed using leave-one-out crossvalidation, resulting in high Pearson and Spearman correlation coefficients for PV-GPe (r=0.42, p<0.0001, ρ=0.52, p<0.0001) and Lhx6-GPe (r=0.42, p<0.0001, ρ=0.41, p<0.0001) models. The generated modulation factors (MF) for PV-GPe and Lhx6-GPe populations at 2.0× threshold intensity are represented as surface plots in FIG. 6, panel B (left and middle). To identify which regions within this parameter space show maximal response separation between PV-GPe and Lhx6-GPe populations, the Lhx6 predictions were subtracted from the PV (FIG. 6, panel B, at right) to determine the population separation index. The models from this first iteration suggested that burst durations near 280 ms, frequencies near 145 Hz, and thresholds near 2.0× threshold would lead to large response differences between the two populations.

To further refine the models, additional experimental data was collected, this time enriching the sampling data for burst parameters falling within the range of values listed in Table 3.

TABLE 3
Parameters Enriched in Iteration 2

| | |
|---|---|
| Frequencies (Hz) | 125, 135, 140, 150, 175, 180, 200 |
| Duration (ms) | 200, 225, 250, 275, 325, 350, 400, 1000, |
| | 4000 |
| Amplitude Multipliers | 1.0, 1.75, 1.9, 2.0, 2.1, 2.15, 2.25, 2.4, 2.5 |

These new data were then combined with the original data set to train a second set of models to predict Lhx6 and PV populations responses to many thousands of artificially generated parameters (Table 2). Because there was strong convergence between the first and second iterations of the models, no additional iterations were run. However, in the enriched data set, Lhx6-GPe neurons were largely stopped (MF_{Lhx6}=−1) for many of the tested combinations, leading the model to behave more as a classifier in the second model iteration for these neurons, resulting in the following Pearson and Spearman correlations (PV-GPe: r=0.48, p<0.0001, ρ=0.57, p<0.0001; Lhx6-GPe r=0.09, p=0.29, ρ=0.51, p<0.0001).

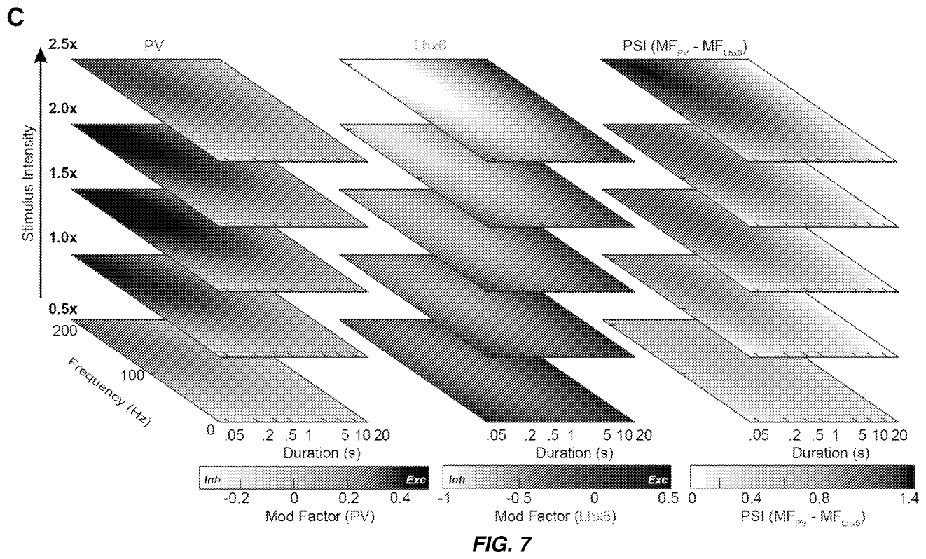

Surface plots across a range of stimulus intensities are shown in FIG. 7 for PV-GPe (left column) and Lhx6-GPe neurons (middle column), as well as for the PSI (right column). Generally, lower amplitude multipliers were predicted to better modulate PV-GPe firing rates than Lhx6-GPe neurons. Burst durations shorter than 1 s were predicted to segregate firing rates, while cell-type specific effects would largely be expected to dissipate 5-10 s into stimulation. Overall, higher intra-burst frequencies were better able to segregate firing responses of PV-GPe and Lhx6-GPe neurons, particularly at frequencies >80 Hz. Additionally, a potential zone for optimizing the segregation of responses between PV-GPe and Lhx6-GPe populations (parameter combinations which resulted in the top 1% of predicted PSIs) was identified: durations near 200 ms (207±157 ms), frequencies near 175 Hz (167±19 Hz), and a stimulus intensity near 2.5× (2.3±0.2) (FIG. 7, right column, top layer, darker shaded region).

### Model-Generated Burst Designs Drive Cell-Type Specific Neuromodulation

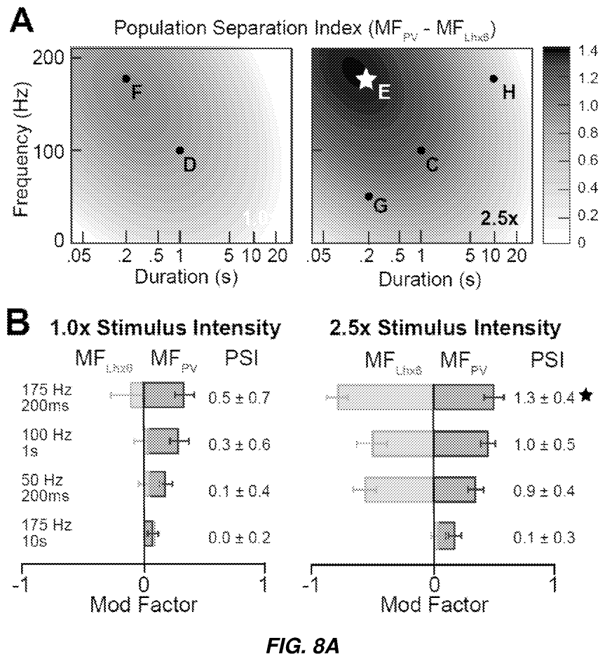

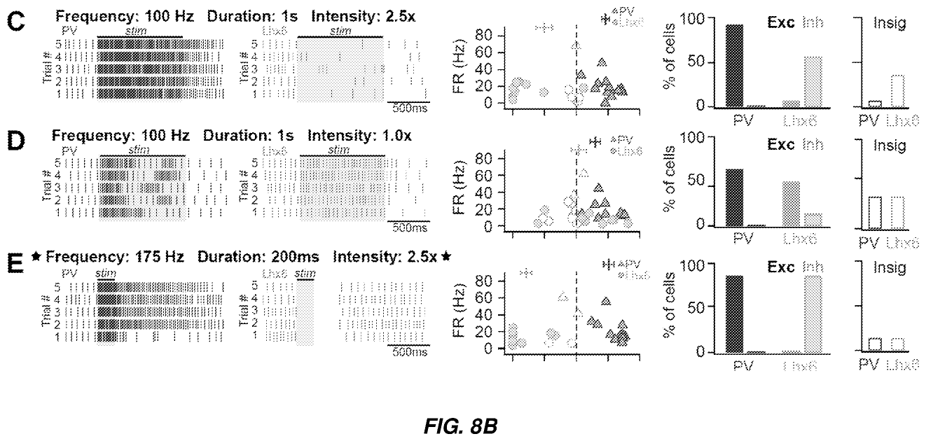

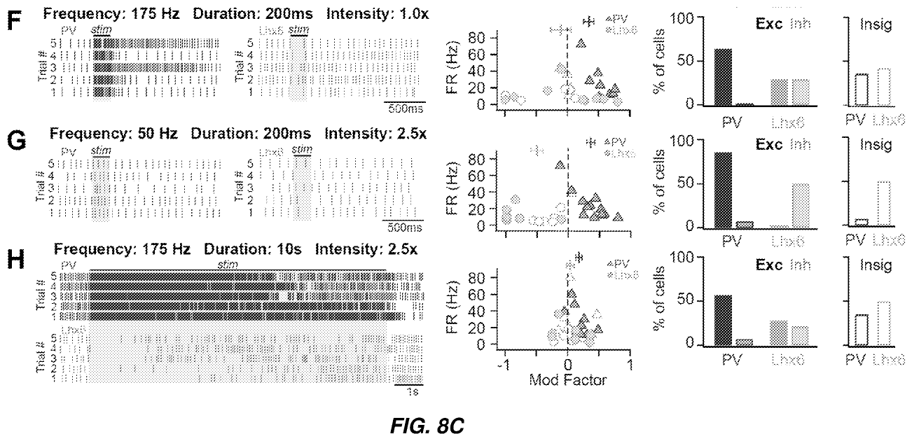

To determine whether the model-based predictions could be implemented to improve the burst design, a baseline selectivity measurement was established using a 100 Hz, 1 s burst design at stimulus intensities of either 1× or 2.5× threshold. The models predict that this burst design should be highly effective at segregating PV-GPe and Lhx6-GPe responses at 2.5×, but less effective at 1× (FIG. 8A, panel A, points C, D). To measure the actual population selectivity obtained experimentally using each burst design, the firing rate responses of in-field pairs of PV-GPe and Lhx6-GPe neurons were measured. Neurons were presented with each burst combination for five trials in order to assess both the magnitude and reliability of their responses. As predicted by the model, 100 Hz, 1 s bursts were highly effective at segregating the responses of PV-GPe and Lhx6-GPe neurons when the stimulation intensity was 2.5× (PSI=0.96±0.5, n=14 pairs across 4 mice) (FIG. 8B, panel C), but not when the stimulation intensity was 1.0× (PSI=0.3±0.6) (FIG. 8B, panel D). Stimulus intensities at 2.5× ranged from 0.075-1.45 mA and stimulus intensities at 1× ranged from 0.03-0.58 mA for these experiments.

To determine whether the cell-type selectivity of the stimulation could be improved even further by refining the burst parameters according to the model, the same PV-GPe and Lhx6-GPe neuron pairs were presented with ‘optimized’ bursts: 175 Hz, 200 ms, 2.5×. Indeed, these bursts proved to be even more effective at segregating the firing responses of PV-GPe and Lhx6-GPe neurons (PSI=1.3±0.4), largely due to an enhancement in the robustness of Lhx6-GPe inhibition (FIG. 8B, panel E). The same burst parameters at 1.0× threshold still retained a small degree of cell-type specificity (PSI=0.5±0.7), but inhibition of Lhx6-GPe neurons was much less reliable (FIG. 8C, panel F). These results illustrate the accuracy of the model's predictions, and establishes burst parameters (175 Hz, 200 ms, 2.5× threshold) that robustly drive cell-type specific neuromodulation using only electrical stimulation.

Another feature of this modeling approach is that it can be used to predict the limitations of parameter combinations that are likely to drive cell-type specific responses. These models suggest that if bursts exceed 10 s, they will be much less effective at driving cell-type specific responses than shorter bursts. To test this prediction, the same PV-GPe and Lhx6-GPe pairs were presented with 175 Hz bursts at 2.5× threshold, but this time, burst duration was 10 s, instead of 200 ms. As shown in FIG. 8C, panel H, 10 s bursts were no longer effective at differentiating the firing responses of PV-GPe and Lhx6-GPe neurons (PSI=0.0±0.2). Consistent with the predictions of the model, stimulating for 10 seconds or longer drives weak excitation in both populations.

This model also predicts limits on the range of intra-burst frequencies that can drive cell type specific responses. Specifically, the model predicts that intra-burst frequencies slower than ˜80 Hz will be less effective at segregating cell-type responses than higher frequencies. To test this prediction, the same PV-GPe and Lhx6-GPe pairs were presented with 200 ms bursts at 2.5× threshold, but with an intra-burst frequency of 50 Hz, instead of 175 Hz. As shown in FIG. 8C, panel G, 50 Hz bursts were still effective at differentiating the firing responses of PV-GPe and Lhx6-GPe neurons (PSI=0.9±0.4), but their efficacy was diminished compared to short, higher frequency bursts.

The results of all parameter combinations tested experimentally are summarized in FIG. 8A, panel B. In general, good correspondence between PSIs measured experimentally and those predicted from the model was seen, particularly at the 2.5× threshold. These experiments show that by using an approach informed by machine learning, the difference in neuromodulation can be strategically maximized by choosing high frequency (175 Hz), short duration bursts (200 ms) (FIG. 8B, panel E, star), resulting in simultaneous excitation of PV-GPe neurons and inhibition of Lhx6-GPe neurons.

### Discussion

In this study, discoveries about the synaptic organization of basal ganglia circuits were applied to develop electrical stimulation protocols that drive cell-type specific neuromodulation in the GPe. Delivering electrical stimulation in short bursts, rather than continuously, augmented natural variations in synaptic inputs onto PV-GPe and Lhx6-GPe neurons, and created transient epochs in which inhibition dominated in Lhx6-GPe neurons and excitation dominated in PV-GPe neurons. This synaptic asymmetry resulted from differences in the degree to which PV-GPe and Lhx6-GPe neurons integrated converging inputs from the STN and striatonigral neurons. Using an active machine learning approach, models were built to predict the non-linear transformations produced by thousands of burst combinations on the firing responses of PV-GPe and Lhx6-GPe populations. Utilizing these predictions, burst parameters were then optimized to maximize cell-type specific neuromodulation.

The results show that the putative optimal pattern to segregate the responses of PV-GPe and Lhx6-GPe populations had an average frequency of 175 Hz and burst duration of 200 ms, at a stimulus intensity 2.5× above slice threshold. These results provide the first demonstration of electrically-driven, cell-type specific neuromodulation in the central nervous system. Electrical stimulation is used in humans to treat a wide variety of conditions including movement disorders, mood and behavioral disorders, and chronic pain (Boccard et al., Deep brain stimulation for chronic pain. J. Clin. Neurosci. 2015, 22: 1537-1543; DeLong et al., Basal ganglia circuits as targets for neuromodulation in Parkinson Disease. JAMA Neurol. 2015, 72: 1354-1360; Formolo et al., Deep brain stimulation for obesity: A review and future directions. Front. Neurosci 2019, 13:323; Holtzheimer et al., Deep brain stimulation for psychiatric disorders. Annu. Rev. Neurosci. 2011, 34: 289-307; Lee et al., Current and future directions of deep brain stimulation for neurological and psychiatric disorders. J. Neurosurg. 2019, 131: 333-342; Xu et al., Deep brain stimulation for Tourette's syndrome. Transl. Neurodegener. 2020, 9: 4; Zhang et al., Mechanisms and models of spinal cord stimulation for the treatment of neuropathic pain. Brain Res. 2014, 1569: 19-31). A number of theoretical approaches have been proposed to improve the cellular- or pathway-specificity of electrical stimulation (Anderson et al., Neural selectivity, efficiency, and dose equivalence in deep brain stimulation through pulse width tuning and segmented electrodes. Brain Stimul. 2020; Mcintyre et al., Extracellular stimulation of central neurons: Influence of stimulus waveform and frequency on neuronal output. J. Neurophysiol. 2002, 88(4): 1592-1604; Mcintyre et al., Modeling the excitability of mammalian nerve fibers: Influence of afterpotentials on the recovery cycle. J. Neurophysiol. 2002, 87: 995-1006; Mcintyre et al., Cellular effects of deep brain stimulation: Model-based analysis of activation and inhibition. J. Neurophysiol. 2004, 91: 1457-1469). In the periphery, there has been some success in translating these approaches into practice, e.g., in the spinal cord and retina (Freeman et al., Selective activation of neuronal targets with sinusoidal electric stimulation. J. Neurophysiol. 2010, 104: 2778-2791; Lempka et al., Innovations in spinal cord stimulation for pain. Curr. Opin. Biomed. Eng. 2018, 8: 51-60), but cell-type selective neuromodulation in the brain has proven to be more challenging (Radman et al., Role of cortical cell type and morphology in subthreshold and suprathreshold uniform electric field stimulation in vitro. Brain Stimul. 2009, 2: 215-228). Strategies to achieve selective stimulation in the brain have focused primarily on varying stimulus waveforms to bias recruitment of fibers with different diameters or degrees of myelination (Grill, Model-based analysis and design of waveforms for efficient neural stimulation. Progress in Brain Research (Elsevier) pp. 147-162 (2015); Grill et al., Stimulus waveforms for selective neural stimulation. IEEE Eng. Med. Biol. Mag. 1995, 14: 375-385; McIntyre et al., Selective microstimulation of central nervous system neurons. Ann. Biomed. Eng. 2000, 28: 219-233), or on generating electric fields that bias recruitment of neurons based on morphology or membrane excitability (Anastassiou et al., The effect of spatially inhomogeneous extracellular electric fields on neurons. J. Neurosci. 2010, 30: 1925-1936). But these methods have not yet achieved the same degree of cell-type specificity as other techniques, such as optogenetics.

Previously, the superior benefits of cell-type specific modulation in recovering motor function after dopamine depletion in a 6-OHDA had been shown in a mouse model of Parkinson's disease (Mastro et al., Cell-specific pallidal intervention induces long-lasting motor recovery in dopamine-depleted mice. Nat. Neurosci. 2017, 20: 815-823). Optogenetic interventions in the GPe that transiently increased the firing rates of PV-GPe neurons and decreased the firing rates of Lhx6-GPe neurons induced a long-lasting recovery of motor function that persisted for hours beyond stimulation. By contrast, optogenetic interventions that globally increased or globally decreased the activity of all GPe neurons were not effective. However, a significant barrier to translating these findings to the clinic was an inability to drive the same degree of cell-type specific modulation using electrical stimulation. The present results represent a significant step forward towards this goal and establish patterns of electrical stimulation that can achieve cell-type specific modulation in the GPe, without the need for optogenetics.

These results are highly relevant for the implementation of DBS in Parkinson's disease. There are few guidelines for programming parameter settings in patients beyond a trial-and error process. Understanding how different patterns of stimulation impact the activity of underlying neural circuits or cell populations would greatly improve the process (Arlotti et al., The adaptive deep brain stimulation challenge. Ark. Relat. Disord. 2016, 28: 12-17; Dorval et al., Deep brain stimulation of the subthalamic nucleus reestablishes neuronal information transmission in the 6-OHDA rat model of parkinsonism. J. Neurophysiol. 2014, 111: 1949-1959; Dorval et al., Deep brain stimulation alleviates parkinsonian bradykinesia by regularizing pallidal activity. J. Neurophysiol. 2010, 104: 911-921; Hashimoto et al., Stimulation of the subthalamic nucleus changes the firing pattern of pallidal neurons. J. Neurosci. 2003, 23(5): 1916-1923; Hess et al., The temporal pattern of stimulation may be important to the mechanism of deep brain stimulation. Exp. Neurol. 2013, 247: 296-302; Montgomery, The epistemology of deep brain stimulation and neuronal pathophysiology. Front. Integr. Neurosci. 2012, 6: 78; Montgomery et al., Mechanisms of action of deep brain stimulation (DBS). Neurosci. Biobehav. Rev. 2008, 32: 388-407; Oza et al., Patterned low-frequency deep brain stimulation induces motor deficits and modulates cortex-basal ganglia neural activity in healthy rats. J. Neuro-Physiol. 2018, 120: 2410-2422). There is currently no consensus about which neural circuits underlie the therapeutic benefits of DBS in Parkinson's disease. One hypothesis is that DBS works by increasing or regularizing the firing rates of STN neurons. Other studies suggest that the therapeutic mechanisms of DBS result from off-target recruitment of other circuits that pass near the STN, emphasizing the non-specific effects of electrical stimulation in vivo. One cannot predict what off-target circuits might be recruited during burst stimulation in vivo, and answers are likely to vary depending on the neuroanatomical features of different species. But the present results strongly suggest that an optimal target for electrode placement would be in an area where fibers from the STN and striatonigral neurons converge. In the experiments in mice, this location was in the internal capsule, between the GPe and STN and in the vicinity of the entopeduncular nucleus (EPN), a rodent equivalent to the internal globus pallidus (GPi). A DBS study in PD patients found that a highly effective site for DBS was the border between GPe and GPi (Vitek et al., Acute stimulation in the external segment of the globus pallidus improves parkinsonian motor signs. Mov. Disord. 2004, 19: 907-915), and posited that a contributing factor to the therapeutic effects of GPe-DBS was co-activation of the direct pathway (referred to as the striatonigral pathway in this study). The prokinetic effects of direct pathway stimulation in Parkinson's disease have been well documented and likely reflect its inhibitory actions on basal ganglia output nuclei. But, under certain stimulation conditions, fibers of the direct pathway can provide an added benefit of driving cell-type specific responses in the GPe. The direct pathway consists of axons of D1-SPN neurons projecting directly from the striatum to basal ganglia output nuclei, the SNr and GPi.

Early anatomical studies of this pathway noted the presence of axon collaterals in the GPe, but without a known functional significance, they had been largely overlooked. The use of optogenetics led to a rediscovery of these collaterals and established their importance for behavioral control and regulating GPe activity. The results reveal a previously unknown cellular specificity of these collaterals in the GPe, where they preferentially inhibit Lhx6-GPe neurons compared to PV-GPe neurons. Striatonigral collaterals might also target another subset of GPe neurons, arkypallidal neurons. Arkypallidal neurons are non-canonical GPe neurons that send a stop signal to the striatum. Although they do not appear to be required for motor rescue in dopamine depleted mice, their inhibition would be predicted to have further prokinetic benefits.

The results presented here provide an opportunity to translate discoveries about the cellular organization of basal ganglia circuits into improved protocols for DBS. Interestingly, clinical work has independently converged upon the study of burst stimulation to improve therapeutic outcomes (Baker et al., Pallidal stimulation: effect of pattern and rate on bradykinesia in the non-human primate model of Parkinson's disease. Exp. Neurol. 2011, 231: 309-313; Brocker et al., Improved efficacy of temporally non-regular deep brain stimulation in Parkinson's disease. Exp. Neurol. 2013, 239: 60-67; Brocker et al., Optimized temporal pattern of brain stimulation designed by computational evolution. Sci. Transl. Med. 2017, 9(371); Grill, Temporal pattern of electrical stimulation is a new dimension of therapeutic innovation. Curr. Opin. Biomed. Eng. 2018, 8: 1-6). One form of burst stimulation, called coordinated reset (CR-DBS) has been shown to provide long-lasting therapeutic effects that extend for days to weeks beyond the end of stimulation (Adamchic et al., Coordinated reset neuromodulation for Parkinson's disease: proof-of-concept study. Mov. Disord. 2014, 29: 1679-1684; Tass et al., Coordinated reset has sustained aftereffects in parkinsonian monkeys. Ann. Neurol. 2012, 72: 816-820; Wang et al., Coordinated reset deep brain stimulation of subthalamic nucleus produces long-lasting, dose-dependent motor improvements in the 1-methyl-4-phenyl-1,2,3,6-tetrahydropyridine non-human primate model of parkinsonism. Brain Stimul. 2016, 9: 609-617). Although its mechanisms of action remain unknown, CR-DBS was developed using principles of spike timing-dependent plasticity to predict stimulus patterns that could train networks out of pathologically synchronized states (Ebert et al., Coordinated reset stimulation in a large-scale model of the STN-GPe circuit. Front. Comput. Neurosci. 2014, 8: 154; Hauptmann et al., Desynchronizing the abnormally synchronized neural activity in the subthalamic nucleus: a modeling study. Expert. Rev. Med. Devices 2007, 4: 633-650; Tass, A model of desynchronizing deep brain stimulation with a demand-controlled coordinated reset of neural subpopulations. Biol. Cybern. 2003, 89: 81-88). CR-DBS is implemented by delivering bursts of stimulation to spatially distributed sites across the STN. The average burst parameters are centered around 150 Hz with durations of 50 ms, strikingly similar to burst parameters identified in the study as being capable of segregating the firing responses of PV-GPe and Lhx6-GPe neurons. Although it is not yet known whether CR-DBS engages similar underlying mechanisms as cell-type specific pallidal interventions, both approaches induce long-lasting motor recovery and persistently reverse pathological firing patterns in the basal ganglia. An advantage of the burst stimulation identified herein is that it can be implemented from a single point source, in contrast to the multi-site stimulation pattern required for CR-DBS, and can be implemented using existing FDA-approved electrode lead designs.

### Example 2

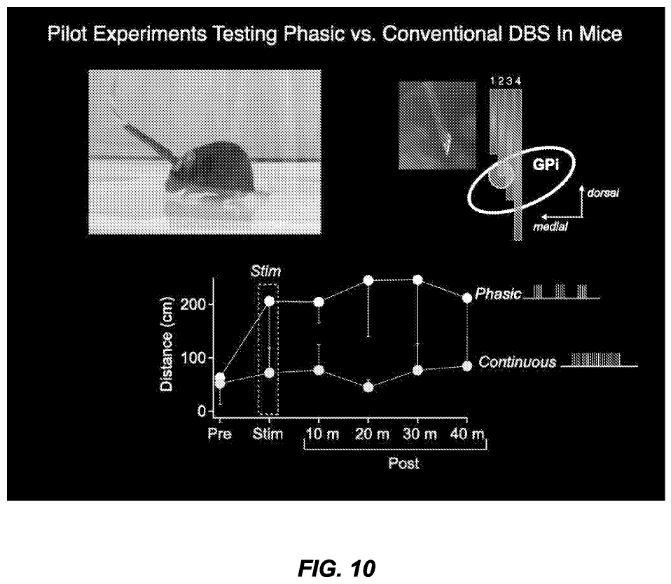

In bilaterally dopamine-depleted mice (depletion induced with 6-OHDA), stimulation delivered bilaterally produced superior and longer-lasting therapeutic benefits when delivered using a burst protocol then when delivered using a conventional, continuous protocol. Stimulation (either burst or continuous) was applied for a total of 30 minutes (‘stim’) and movement was tracked throughout the experiment. Continuous stimulation consisted of 175 Hz stimulation (bipolar stimulation, 100 us pulse width) for the full 30 minutes. Burst stimulation consisted of 175 Hz stimulation (bipolar stimulation, 60 us pulse width) delivered for 250 ms, then off for 750 ms, then again for 250 ms, off for 250 ms, etc for 30 minutes. Electrodes were targeted to the white matter track between the GP and STN. As shown in FIG. 10, while continuous stimulation resulted in a very mild improvement in distance moved, burst stimulation resulted in a significantly greater restoration of motor impairment. In addition, the restoration of motor impairment can be seen to persist for at least 40 minutes after the burst stimulation was discontinued. These results suggest that when used in human patients with Parkinson's disease, the effects of burst stimulation could not only be effective at symptom relief, but that relief could persist beyond the time period associated with continuous stimulation. Clinical benefits could include fewer off-target side effects generated by DBS and preservation of battery life.

Although the invention has been described in detail for the purpose of illustration based on what is currently considered to be the most practical and preferred embodiments, it is to be understood that such detail is solely for that purpose and that the invention is not limited to the disclosed embodiments, but on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A method of modulating a plurality of neurons in a patient, comprising:
stimulating an area of the patient's central nervous system, the stimulation comprising alternating first periods when a plurality of pulses of electrical stimulation are delivered and second periods when no pulses of electrical stimulation are delivered,
wherein the first periods have a duration of about 100 to about 400 ms and the second periods have a duration of about 500 ms to about 1900 ms, and
wherein the pulses have a frequency of about 100 Hz to about 250 Hz.

2. (canceled)

3. (canceled)

4. The method according to claim 1, wherein the area of the patient's central nervous system is the internal globus pallidus or the external globus pallidus.

5. (canceled)

6. (canceled)

7. The method according to claim 1, wherein the first periods have a duration of about 150 ms to about 250 ms.

8. The method according to claim 1, wherein the pulses have a frequency of about 150 Hz to about 175 Hz.

9. (canceled)

10. The method according to claim 1, wherein the pulses have an amplitude of about 0.1 to about 4 mA.

11. The method according to claim 1, wherein the pulses are biphasic pulses.

12. (canceled)

13. (canceled)

14. The method according to claim 1, wherein the globus pallidus of each hemisphere is stimulated.

15. The method of claim 1, wherein the area of the patient's central nervous system is the basal ganglia the patient has a movement disorder the stimulation is of the patient's basal ganglia, and wherein the stimulation improves one or more motor symptoms of the movement disorder.

16. (canceled)

17. The method according to claim 15, wherein only a single stimulating electrode lead is used, and the stimulating electrode lead is implanted in the patient's external globus pallidus or internal globus pallidus.

18. (canceled)

19. (canceled)

20. The method according to claim 15, wherein the first periods have a duration of about 150 ms to about 250 ms.

21. The method according to claim 15, wherein the pulses have a frequency of about 150 Hz to about 175 Hz.

22. (canceled)

23. The method according to claim 15, wherein the pulses have an amplitude of about 0.1 to about 4 mA.

24. The method according to claim 15, wherein the pulses are biphasic pulses.

25. The method according to claim 15, wherein the movement disorder is selected from Parkinson's Disease, essential tremor, dystonia, and Meige Syndrome.

26. (canceled)

27. (canceled)

28. The method according to claim 15, wherein two stimulating electrode leads are implanted, and wherein one electrode lead is implanted in the globus pallidus of each hemisphere.

29. A system for modulating a plurality of neurons in a patient, comprising:
an electrode lead;
a pulse generator in electrical communication with the electrode lead, the pulse generator comprising at least one processor programmed or configured to cause the pulse generator to deliver stimulation through the electrode lead,
wherein the stimulation comprises alternating first periods when a plurality of pulses of electrical stimulation are delivered and second periods when no pulses of electrical stimulation are delivered,
wherein the first periods have a duration of about 100 to about 400 ms and the second periods have a duration of about 500 ms to about 1900 ms, and
wherein the pulses have a frequency of about 100 Hz to about 250 Hz.

30. The system according to claim 29, wherein the pulse generator further includes memory and a communication interface.

31. (canceled)

32. (canceled)

33. (canceled)

34. The system according to claim 29, wherein the first periods have a duration of about 150 ms to about 250 ms.

35. The system according to claim 29, wherein the pulses have a frequency of about 150 Hz to about 175 Hz.

36. (canceled)

37. The system according to claim 29, wherein the pulses have an amplitude of/intensity of about 0.1 to about 4 mA.

38. (canceled)

39. (canceled)

40. (canceled)
